(19) United States
(12) Patent Application Publication
(10) Pub. No.: US 2016/0113480 A1
(43) Pub. Date: Apr. 28, 2016

(54) DECODING DEVICE, IMAGING SYSTEM, DECODING METHOD, CODING/DECODING METHOD, AND COMPUTER-READABLE RECORDING MEDIUM

(71) Applicant: OLYMPUS CORPORATION, Tokyo (JP)

(72) Inventors: Masatoshi HOMAN, Tokyo (JP); Kenichi TAKIZAWA, Yokohama-shi (JP)

(73) Assignee: OLYMPUS CORPORATION, Tokyo (JP)

(21) Appl. No.: 14/992,485

(22) Filed: Jan. 11, 2016

Related U.S. Application Data
(63) Continuation of application No. PCT/JP2015/058131, filed on Mar. 18, 2015.

(30) Foreign Application Priority Data
Sep. 3, 2014 (JP) 2014-179366

(51) Int. Cl. A61B 1/00; H04N 19/172; H04N 19/423; A61B 1/04; H04N 19/65; H04N 19/117; H04N 19/186; H04N 9/04; H04N 19/176; H04N 7/18

(52) U.S. Cl. CPC A61B 1/00009; H04N 19/176; H04N 19/172; H04N 19/423; H04N 7/185; H04N 19/65; H04N 19/117; H04N 19/186; H04N 9/045; A61B 1/041

(57) ABSTRACT

A decoding device includes: a data obtaining unit configured to obtain a key block and a non-key brock, the key block forming a part of image data of one frame, and the non-key block forming a part of the image data of one frame on at least a part of which a coding process is performed; a characteristic information storage unit configured to store characteristic information regarding a pixel value correlation characteristic in a frame; and a decoding unit configured to perform repeated decoding by a belief-propagation method based on a first log-likelihood ratio and a second log-likelihood ratio to estimate the non-key block before the coding process, the first log-likelihood being obtained from the non-key block, and the second log-likelihood ratio being obtained from the key block and the characteristic information.

### CROSS-REFERENCE TO RELATED APPLICATIONS

This application is a continuation of PCT international application Ser. No. PCT/JP2015/058131 filed on Mar. 18, 2015 which designates the United States, incorporated herein by reference, and which claims the benefit of priority from Japanese Patent Application No. 2014-179366, filed on Sep. 3, 2014, incorporated herein by reference.

### BACKGROUND

1. Technical Field

The present invention relates to a decoding device which decodes image data coded by an imaging device, an imaging system, a decoding method, a coding/decoding method, and a computer-readable recording medium.

2. Related Art

Conventionally, a system using a swallow type capsule endoscope is suggested, for example, as an imaging system provided with an imaging device which transmits image data generated by capturing an image of a subject and a receiving device which receives the image data (for example, see Japanese Patent Application Laid-open No. 2006-293237).

Such capsule endoscope moves in a body cavity, for example, in an organ such as stomach and small intestine by peristalsis and captures an in-vivo image of the subject at each predetermined time while this moves after this is swallowed from mouth of the subject for observation (examination) until spontaneous excretion.

The capsule endoscope sequentially transmits image data captured in-vivo to outside by wireless communication while this moves in the body cavity.

### SUMMARY

In some embodiments, a decoding device configured to decode image data coded by an imaging device is presented. The decoding device includes: a data obtaining unit configured to obtain a key block and a non-key brock, the key block forming a part of the image data of one frame generated by the imaging device, and the non-key block forming a part of the image data of one frame generated by the imaging device on at least a part of which a coding process is performed; a characteristic information storage unit configured to store characteristic information regarding a pixel value correlation characteristic in a frame; and a decoding unit configured to perform repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and the characteristic information stored in the characteristic information storage unit.

In some embodiments, an imaging system includes an imaging device configured to code image data generated by capturing an image of a subject to transmit, and a decoding device configured to receive the coded image data to decode. The imaging device includes: an imaging unit configured to generate image data corresponding to incident light and sort the image data into a key block and a non-key block for each frame; a coding unit configured to perform a coding process on at least a part of the non-key block; and a transmitting unit configured to transmit the key block and the non-key block on at least a part of which the coding process is performed. The decoding device includes: a receiving unit configured to receive the key block and the non-key block on at least a part of which the coding process is performed; a characteristic information storage unit configured to store characteristic information regarding a pixel value correlation characteristic for each color in a frame; and a decoding unit configured to perform repeated decoding by a belief-propagation method based on a first log-likelihood ratio and a second log-likelihood ratio to estimate the non-key block before coding process, the first log-likelihood being obtained from the non-key block on at least a part of which the coding process is performed, and the second log-likelihood ratio being obtained from the key block and the characteristic information stored in the characteristic information storage unit.

In some embodiments, a decoding method executed by a decoding device configured to decode image data coded by an imaging device is presented. The decoding method includes: obtaining a key block and a non-key brock, the key block forming a part of the image data of one frame generated by the imaging device, and the non-key block forming a part of the image data of one frame generated by the imaging device on at least a part of which a coding process is performed; and performing repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic in a frame.

In some embodiments, a coding and decoding method executed by an imaging system including an imaging device configured to code image data generated by capturing an image of a subject to transmit and a decoding device configured to receive the coded image data to decode is presented. The coding and decoding method includes: generating, by the imaging device, image data corresponding to incident light; sorting, by the imaging device, the image data into a key block and a non-key block for each frame; performing, by the imaging device, a coding process on at least a part of the key block; transmitting, by the imaging device, the key block and the non-key block on at least a part of which the coding process is performed; receiving, by the decoding device, the key block and the non-key block on at least a part of which the coding process is performed; and performing, by the decoding device, repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic for each color in a frame.

In some embodiments, a non-transitory computer-readable recording medium having an executable program recorded thereon is presented. The program instructs a processor, which is included in a decoding device configured to decode image data coded by an imaging device, to execute: obtaining a key block and a non-key brock, the key block forming a part of the image data of one frame generated by the imaging device, and the non-key block forming a part of the image data of one frame generated by the imaging device on at least a part of which a coding process is performed; and

performing repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic in a frame.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating an imaging system according to a first embodiment of the present invention;

FIG. 2 is a view illustrating an example of a key block and a non-key block according to the first embodiment of the present invention;

FIG. 3 is a view for illustrating a coding process according to the first embodiment of the present invention;

FIG. 4A is a view illustrating an example of characteristic information according to the first embodiment of the present invention;

FIG. 4B is a view illustrating an example of the characteristic information according to the first embodiment of the present invention;

FIG. 5 is a view for illustrating an example of repeated decoding (belief-propagation method) according to the first embodiment of the present invention;

FIG. 6 is a flowchart illustrating a coding/decoding method according to the first embodiment of the present invention;

FIG. 7 is a flowchart illustrating a decoding process according to the first embodiment of the present invention;

FIG. 8 is a flowchart illustrating a coding/decoding method according to a second embodiment of the present invention;

FIG. 9 is a block diagram illustrating an imaging system according to a third embodiment of the present invention;

FIG. 10 is a flowchart illustrating a coding/decoding method according to the third embodiment of the present invention;

FIG. 11 is a block diagram illustrating an imaging system according to a fourth embodiment of the present invention;

FIG. 12 is a view virtually illustrating a function of a sorting unit according to the fourth embodiment of the present invention;

FIG. 13 is a flowchart illustrating a coding/decoding method according to the fourth embodiment of the present invention;

FIG. 14 is a schematic diagram illustrating a capsule endoscope system according to a fifth embodiment of the present invention;

FIG. 15 is a block diagram illustrating a decoding device according to the fifth embodiment of the present invention;

FIG. 16 is a view illustrating a variation of the first to fifth embodiments of the present invention; and

FIG. 17 is a view illustrating a variation of the first to fifth embodiments of the present invention.

### DETAILED DESCRIPTION

A preferred embodiment of a decoding device, an imaging system, a decoding method, a coding/decoding method, and a decoding program according to the present invention is hereinafter described in detail with respect to the drawings. Meanwhile, the present invention is not limited by the embodiment.

### First Embodiment

### Schematic Configuration of Imaging System

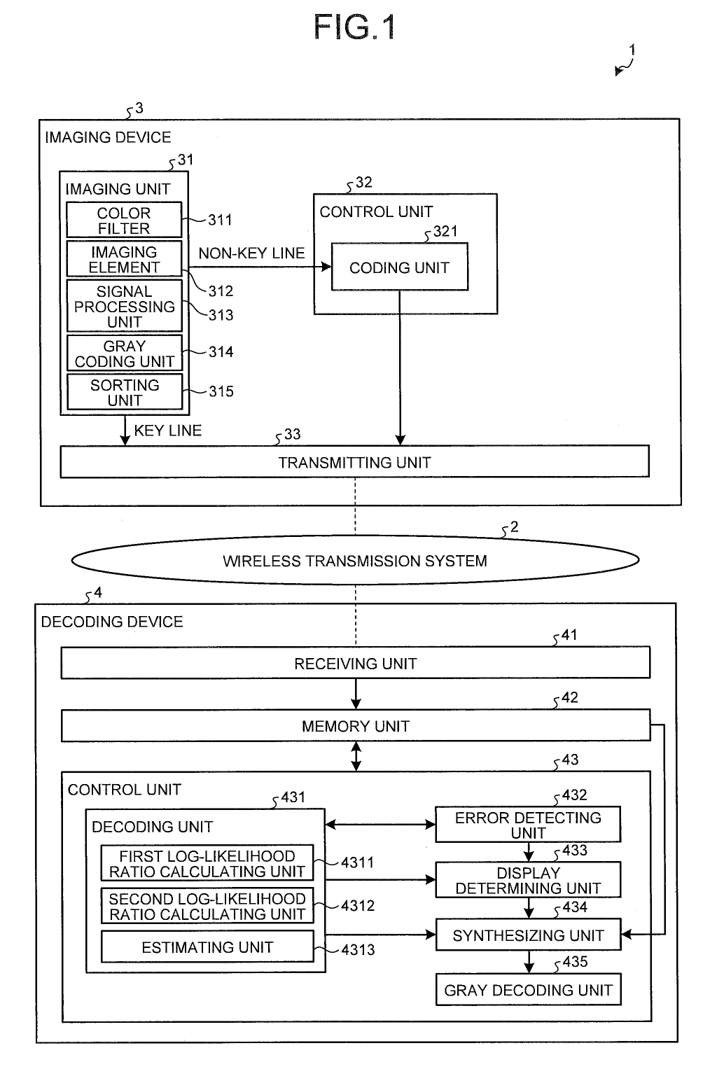

FIG. 1 is a block diagram illustrating an imaging system 1 according to a first embodiment of the present invention.

The imaging system 1 is provided with an imaging device 3 and a decoding device 4, which wirelessly communicate moving image data through a wireless transmission system 2 as illustrated in FIG. 1.

Configuration of Imaging Device

The imaging device 3 codes the moving image data generated by capturing an image of a subject and wirelessly transmits the same through the wireless transmission system 2. The imaging device 3 is provided with an imaging unit 31, a control unit 32, a transmitting unit 33 and the like.

The imaging unit 31 captures an image of the subject at a frame rate of 30 frames per second, for example, to sequentially generate image data and sorts the image data into a key block and a non-key block for each frame under control of the control unit 32. The imaging unit 31 is provided with a color filter 311, an imaging element 312, a signal processing unit 313, a Gray coding unit 314, a sorting unit 315 and the like.

The color filter 311 arranged on a light receiving surface of the imaging element 312 has a configuration in which a plurality of filter groups grouped according to wavelength bands of light which passes therethrough is arranged in a specified format (for example, Bayer array).

Hereinafter, the color filter 311 is described as the color filter 311 of the Bayer array, that is to say, the color filter 311 including a red filter group through which light of a red wavelength band passes, a blue filter group through which light of a blue wavelength band passes, a first green filter group (arranged in the same column as the red filter group) through which light of a green wavelength band passes, and a second green filter group (arranged in the same column as the blue filter group) through which the light of the green wavelength band passes.

The imaging element 312 is driven by an imaging element driving circuit (not illustrated) and converts incident light through the color filter 311 to an electric signal to form an image. The imaging element driving circuit obtains image data of an analog signal by driving the imaging element 312 and outputs the image data of the analog signal to the signal processing unit 313.

The signal processing unit 313 generates digital image data by performing specified signal processing such as sampling, amplification, and A/D (analog to digital) conversion, for example, on the image data of the analog signal output from the imaging element 312 and outputs the same to the Gray coding unit 314.

The Gray coding unit 314 performs Gray coding on the image data (moving image frame sequence) from the signal processing unit 313. For example, the Gray coding unit 314 performs the Gray coding on a pixel value “6 (“0110” in binary representation)”, a pixel value “7 (“0111” in binary representation)”, and a pixel value “8 (“1000” in binary representation)” of pixels of the image data to obtain Gray codes “0101”, “0100”, and “1100”, respectively. The Gray code has a characteristic that the data always changes by only one bit when changing from a certain value to an adjacent value.

The sorting unit 315 sorts the image data (moving image frame sequence) Gray coded by the Gray coding unit 314 into the key block and the non-key block for each frame.

Herein, although the Gray coding unit 314 performs the Gray coding on the image data from the signal processing unit 313, there is no limitation; it is also possible to perform the Gray coding only on the non-key block sorted by the sorting unit 315. That is to say, the Gray coding of the key block is not indispensable.

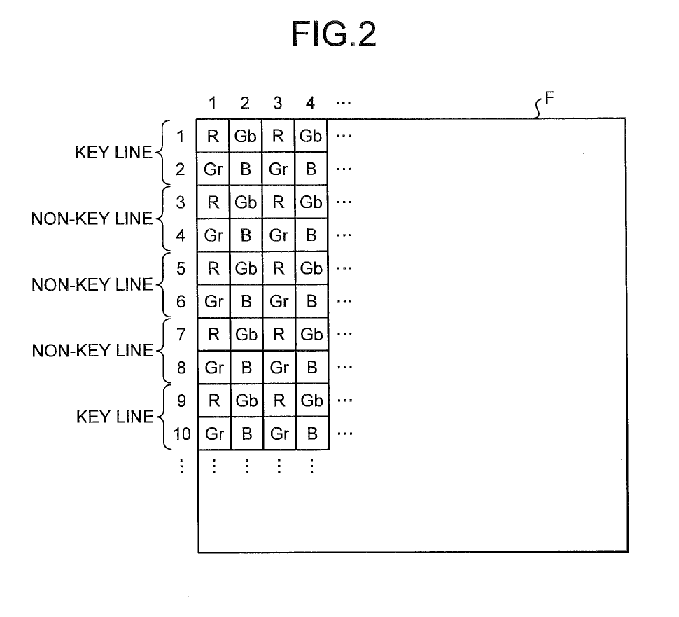

FIG. 2 is a view illustrating an example of the key block and the non-key block according to the first embodiment of the present invention. Meanwhile, in FIG. 2, a reference sign “R” is assigned to each red pixel of a red-corresponding pixel group corresponding to the red filter group of the color filter 311, a reference sign “B” is assigned to each blue pixel of a blue-corresponding pixel group corresponding to the blue filter group, a reference sign “Gr” is assigned to each first green pixel of a first green-corresponding pixel group corresponding to the first green filter group, and a reference sign “Gb” is assigned to each second green pixel of a second green-corresponding pixel group corresponding to the second green filter group in an image F of one frame.

Specifically, a plurality of pixels of two rows out of a plurality of pixels arranged in a matrix pattern is made one block, and the sorting unit 315 sorts such that one out of several blocks is made the key block (hereinafter, referred to as a key line) and the others are made the non-key blocks (hereinafter referred to as non-key lines) in ascending order of row numbers for each frame as illustrated in FIG. 2. In an example in FIG. 2, the sorting unit 315 makes one out of four blocks the key line.

Then, the sorting unit 315 outputs the key line to the transmitting unit 33 and outputs the non-key line to the control unit 32.

The control unit 32 including a CPU (central processing unit) and the like controls operation of an entire imaging device 3. The control unit 32 is provided with a coding unit 321 and the like.

The coding unit 321 to which the non-key line from the sorting unit 315 is sequentially input performs a coding process on each non-key line. Then, the coding unit 321 sequentially outputs the non-key line on which the coding process is performed to the transmitting unit 33.

Hereinafter, in order to specifically describe the coding process by the coding unit 321, the description focuses on one pixel included in the non-key line.

Specifically, when the Gray code (bit sequence) of one pixel in the input non-key line is xᵢ, the coding unit 321 performs syndrome coding by using a low-density parity check matrix H of (n−k) rows by n columns as represented by following equation (1). Then, the coding unit 321 sequentially outputs the non-key line after the coding process (syndrome C) to the transmitting unit 33.

Herein, when the check matrix H of (n−k) rows by n columns is used, a coding rate is k/n and a compression rate is (n−k)/n.

C=H xᵢ (1)

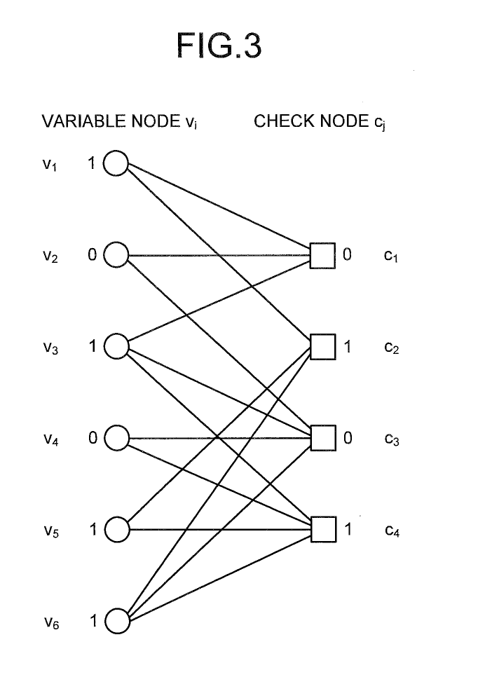

FIG. 3 is a view for illustrating the coding process according to the first embodiment of the present invention. Meanwhile, in FIG. 3, a case in which the low-density parity check matrix H of four rows by six columns (n=6, k=2) represented by following equation (2) is used as the low-density parity check matrix H (input Gray code xᵢ is of six bits) is illustrated.

$$H = [ \begin{matrix}1 & 1 & 1 & 0 & 0 & 0 \\ 1 & 0 & 0 & 0 & 1 & 1 \\ 0 & 1 & 1 & 1 & 0 & 1 \\ 0 & 0 & 1 & 1 & 1 & 1\end{matrix} ] \qquad ( 2 )$$

The check matrix H may be represented by a bipartite graph indicating a connection state between a plurality of variable nodes one-to-one corresponding to a plurality of columns and a plurality of check nodes one-to-one corresponding to a plurality of rows as illustrated in FIG. 3.

Specifically, in the bipartite graph, a plurality of variable nodes vᵢ (i=1 to 6 in the example in FIG. 3) one-to-one corresponding to a plurality of columns of the check matrix H is arranged on a left side. A plurality of check nodes cⱼ (j=1 to 4 in the example in FIG. 3) one-to-one corresponding to a plurality of rows of the check matrix H is arranged on a right side. The variable node vᵢ and the check node cⱼ corresponding to combination of the row and column with which a component of the check matrix H is “1” are connected to each other by a line (referred to as edge).

For example, in the example in FIG. 3, a variable node v₂ corresponding to a second column and a check node c₃ corresponding to a third row are connected to each other by the edge; this indicates that a third row-second column component of the check matrix H is “1”.

It is possible to easily perform the syndrome coding represented by equation (1) by using such bipartite graph.

For example, when the Gray code xᵢ (of six bits in the example in FIG. 3) of one pixel subjected to the coding process is “101011”, the Gray code xᵢ is assigned to each of the variable nodes vᵢ as illustrated in FIG. 3. Then, focusing on each of the check nodes cⱼ, binary addition of all the variable nodes vᵢ connected thereto by the edges is performed. For example, when focusing on a check node c₁, the variable nodes vᵢ connected to the check node c₁ by the edges are the variable nodes v₁, v₂, and v₃, so that the binary addition of the values “1”, “0”, and “1” of the variable nodes v₁, v₂, and v₃, respectively, is performed to obtain a value of “0”. Then, “0101” calculated at respective check nodes cⱼ becomes the syndrome C.

That is to say, when the low-density parity check matrix H as represented by equation (2) is used, the six-bit Gray code xᵢ is compressed to a four-bit syndrome C (at a compression rate of ⅔).

Meanwhile, as the low-density parity check matrix H, the check matrix of the coding rate of ⅓ and the compression rate of ⅔ as represented by equation (2) or that of the compression rate of ⅓ may also be adopted. The check matrix of the compression rate of 33 to 50% is preferably adopted.

The transmitting unit 33 makes the key line from the sorting unit 315 and the non-key line after the coding process (syndrome C) from the coding unit 321 a data stream under control of the control unit 32. The transmitting unit 33 transmits the moving image data which is made the data stream to the decoding device 4 through the wireless transmission system 2.

Configuration of Decoding Device

The decoding device 4 receives the moving image data (data stream) transmitted from the imaging device 3 through the wireless transmission system 2 to decode. The decoding device 4 is provided with a receiving unit 41, a memory unit 42, a control unit 43 and the like as illustrated in FIG. 1.

The receiving unit 41 is formed of an antenna and the like for receiving the moving image data transmitted from the imaging device 3 through the wireless transmission system 2. The receiving unit 41 sequentially receives the moving image data to output to the memory unit 42 under control of the control unit 43.

Meanwhile, the moving image data received by the receiving unit 41 is hereinafter referred to as received data for the purpose of description.

The above-described receiving unit 41 serves as a receiving unit according to the present invention and also serves as a data obtaining unit according to the present invention.

The memory unit 42 sequentially stores the received data output from the receiving unit 41. The memory unit 42 stores various programs (including a decoding program) executed by the control unit 43 and information required for a process of the control unit 43 and the like. Furthermore, the memory unit 42 stores characteristic information regarding a pixel value correlation characteristic in the frame or the pixel value correlation characteristic for each color in the frame. That is to say, the memory unit 42 serves as a characteristic information storage unit according to the present invention.

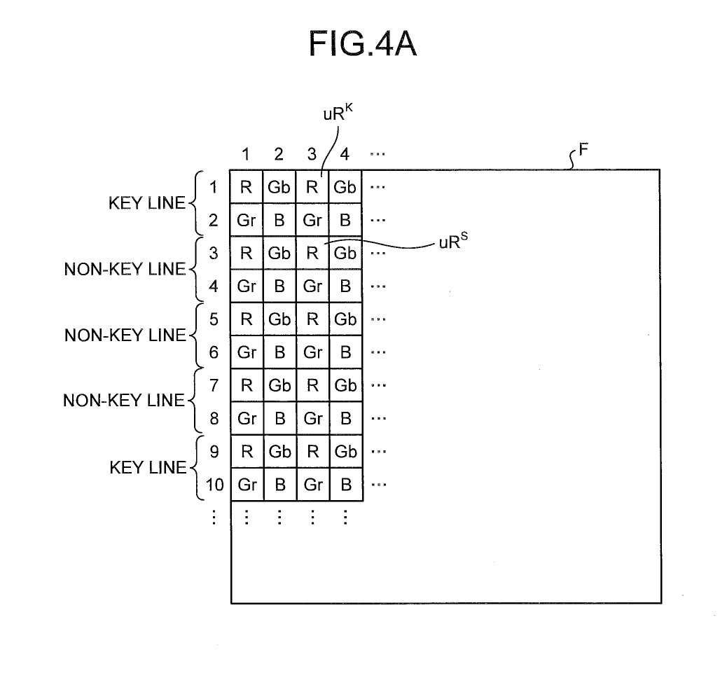

FIGS. 4A and 4B are views illustrating an example of the characteristic information according to the first embodiment of the present invention.

The characteristic information calculated from the image data generated by capturing in advance is the information indicating how the pixel value (Gray code) changes in the frame by probability distribution.

Meanwhile, in the first embodiment of the present invention, the memory unit 42 divides the image data captured in advance into the corresponding pixel groups (red-corresponding pixel group, blue-corresponding pixel group, first green-corresponding pixel group, and second green-corresponding pixel group) corresponding to the filter groups of the color filter 311 and stores the characteristic information for red pixel, blue pixel, first green pixel, and second green pixel calculated for each of the corresponding pixel groups as the above-described characteristic information.

For example, as illustrated in FIG. 4A, in the frame, the pixel value (Gray code (illustrated by four bits)) of one red pixel R (first row-third column red pixel R) included in the block serving as the key line is set to uR^{K} (uR^{K}=5 (Gray code is “0111”) in the example in FIGS. 4A and 4B) and the pixel value (Gray code (illustrated by four bits)) of one red pixel R (third row-third column red pixel R) included in the block having a larger row number than that of this block located in an adjacent position (block serving as the non-key line), the red pixel the closest to the above-described red pixel R is set to uR^{S}. In this case, the characteristic information illustrated in FIG. 4B is stored in the memory unit 42 as the characteristic information for red pixel between the pixel values uR^{K} and uR^{S}.

Specifically, the characteristic information for red pixel between the pixel values uR^{K} and uR^{S} is the information obtained by approximating probability P(uR^{S}) which the pixel value uR^{S} might take by Laplace distribution as illustrated in FIG. 4B.

Meanwhile, the probability P(uR^{S}) which the pixel value uR^{S} might take may be obtained by using other than the Laplace distribution.

The pixel value uR^{S} and the probability P(uR^{S}) which this might take illustrated in FIG. 4B are summarized in following Table 1.

That is to say, as illustrated in FIG. 4B and Table 1, the probability P(uR^{S}) of “5” the same as the pixel value uR^{K} is the highest (50%) and the probability P(uR^{S}) decreases as the pixel value separates from the pixel value uR^{K}.

TABLE 1

| | pixel value uR^{s} | | | | |
|---|---|---|---|---|---|
| | 1(“0001”) | 2(“0011”) | 3(“0010”) | 4(“0110”) | |
| | 9(“1101”) | 8(“1100”) | 7(“0100”) | 6(“0101”) | 5(“0111”) |
| Probability | 0% | 2.5% | 5% | 12.5% | 50% |
| P(uR^{s}) | | | | | |

The control unit 43 including a CPU and the like reads the program (including the decoding program) stored in the memory unit 42 and controls operation of an entire decoding device 4 according to the program. The control unit 43 is provided with a decoding unit 431, an error detecting unit 432, a display determining unit 433, a synthesizing unit 434, a Gray decoding unit 435 and the like as illustrated in FIG. 1.

The decoding unit 431 performs repeated decoding by a belief-propagation method by using first and second two log-likelihood ratios (likelihood change between the first and second log-likelihood ratios) and performs a decoding process to estimate the non-key line before the coding process by the imaging device 3. The decoding unit 431 is provided with a first log-likelihood ratio calculating unit 4311, a second log-likelihood ratio calculating unit 4312, an estimating unit 4313 and the like.

Meanwhile, the decoding unit 431 performs the process in block on all the pixels included in the non-key line when estimating the non-key line before the coding process.

Hereinafter, in order to specifically describe the repeated decoding by the decoding unit 431, the description focuses on one pixel included in the non-key line. Hereinafter, for the purpose of description, the frame to be decoded is referred to as a target frame, the non-key line to be decoded in the target frame is referred to as a target line, and the pixel to be decoded in the target line is referred to as a target pixel.

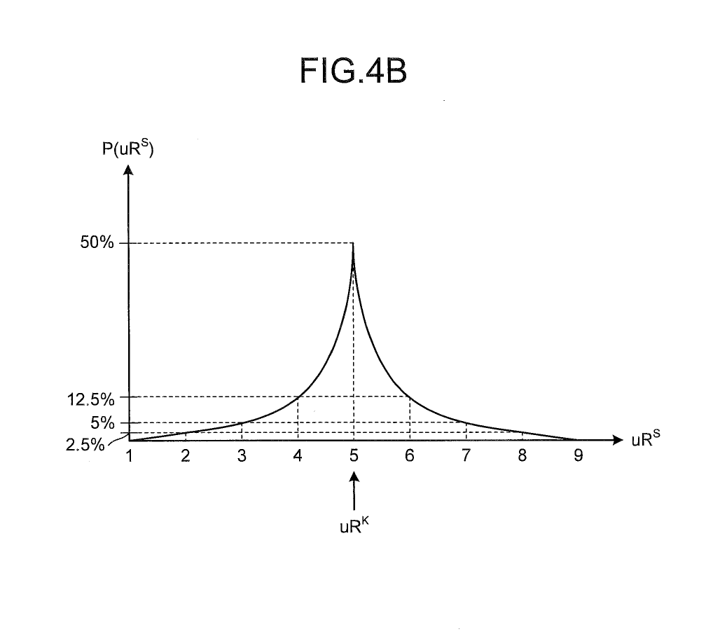

FIG. 5 is a view for illustrating the repeated decoding (belief-propagation method) according to the first embodiment of the present invention. Meanwhile, in FIG. 5, for the purpose of description, only one variable node vᵢ and only one check node cⱼ (refer to FIG. 3, for example) are illustrated. In FIG. 5, a subscript “wᵢ” represents the number of edges connected to an i-th variable node vᵢ. Similarly, a subscript “rⱼ” represents the number of edges connected to a j-th check node cⱼ.

Specifically, the decoding unit 431 performs the repeated decoding to repeat a specified number of times the likelihood change to transmit a second log-likelihood ratio q_{i,m} from the variable node vᵢ to the check node cⱼ along an m-th (m=1 to wᵢ) edge of the variable node vᵢ and transmit a first log-likelihood ratio t_{j,m′} from the check node cⱼ to the variable node vᵢ along an m′-th (m′=1 to rⱼ) edge of the check node cⱼ on the bipartite graph representing the low-density parity check matrix H of (n−k) rows by n columns used in the syndrome coding by the imaging device 3 as illustrated in FIG. 5.

Meanwhile, in FIG. 5, for the purpose of description, the second log-likelihood ratio q_{i,m} output from the variable node vᵢ along the m-th edge of the variable node vᵢ is represented with a superscript “out”. The log-likelihood ratio (first log-likelihood ratio t_{j,m′}) which enters the variable node vᵢ along the m-th edge of the variable node vᵢ is represented as q_{i,m} with a superscript “in”. Similarly, the first log-likelihood ratio t_{j,m′} exiting from the check node cⱼ along the m′-th edge of the check node cⱼ is represented with a superscript “out”. The log-likelihood ratio (second log-likelihood ratio q_{i,m}) which enters the check node cⱼ along the m′-th edge of the check node is represented as t_{j,m′} with the superscript “in”.

Herein, the log-likelihood ratio (LLR) is obtained by taking a log of a ratio of probability P(0) with which a certain bit is “0” and probability P(1) with which this is “1” as represented by following equation (3). When the log-likelihood ratio is not smaller than 0, it may be evaluated that the bit corresponding to a value of the log-likelihood ratio is “0”, and when the log-likelihood ratio is smaller than 0, it may be evaluated that the bit corresponding to the value of the log-likelihood ratio is “1”. It is possible to evaluate whether the value of the bit corresponding to the value of the log-likelihood ratio is “0” or “1” with higher reliability as an absolute value of the log-likelihood ratio is larger.

In equation (3), γ is a parameter to correct the log-likelihood ratio having a positive actual number larger than 0; values such as “1.0” and “0.4” are appropriately used for the first log-likelihood ratio and the second log-likelihood ratio, respectively, for example.

$$L L R = γ log \frac{P ( 0 )}{P ( 1 )} \qquad ( 3 )$$

The second log-likelihood ratio calculating unit 4312 first reads the key line which comes immediately before the target line (non-key line) in chronological order in the target frame (with a smaller row number adjacently located in the target frame) and the characteristic information corresponding to types of the target pixels (red pixel, blue pixel, first green pixel, and second green pixel) included in the target line from the memory unit 42 and calculates a second log-likelihood ratio q_{i,0} serving as an initial value of the second log-likelihood ratio q_{i,m}. Then, the second log-likelihood ratio calculating unit 4312 transmits the calculated second log-likelihood ratio q_{i,0} from the variable node vᵢ to the check node cⱼ along the edge in first likelihood change.

For example, when the target pixel of the target line (non-key line) is the red pixel R (third row-third column red pixel R) illustrated in FIG. 4A, and when the pixel value of the red pixel R (first row-third column red pixel R) the closest to the target pixel in the key line read from the memory unit 42 is “5” (the example in FIGS. 4A and 4B), the second log-likelihood ratio calculating unit 4312 calculates the second log-likelihood ratio q_{i,0} (i=1 to 4) serving as an initial value of the pixel value uR^{S} of the target pixel as described below.

A highest-order bit of the pixel value uR^{S} is “0” when the pixel value uR^{S} is “1(“0001”)”, “2(“0011”)”, 3(“0010”)”, “4(“0110”)”, “5(“0111”)”, “6(“0101”)”, and “7(“0100”)”. Therefore, it is possible to calculate the probability P(0) from the probability P(uR^{S}) in the above-described case based on the characteristic information for red pixel illustrated in FIG. 4B and Table 1.

On the other hand, the highest-order bit of the pixel value uR^{S} is “1” when the pixel value uR^{S} is “8(“1100”)” and “9(“1101”)”. Therefore, it is possible to calculate the probability P(1) from the probability P(uR^{S}) in the above-described case based on the characteristic information for red pixel illustrated in FIG. 4B and Table 1.

When the probabilities P(0) and P(1) are calculated in the above-described manner, it is possible to calculate a second log-likelihood ratio q_{1,0} of the first bit of the target pixel by equation (3).

Meanwhile, each of second log-likelihood ratios q_{2,0} to q_{4,0} of second, third, and fourth highest-order bits of the target pixel may also be calculated in the similar manner.

The second log-likelihood ratio calculating unit 4312 transmits the calculated second log-likelihood ratios q_{1,0} to q_{4,0} from the variable nodes v₁ to v₄, respectively.

The second log-likelihood ratio calculating unit 4312 updates the second log-likelihood ratio q_{i,m} by following equation (4) during the likelihood change between the first and second log-likelihood ratios performed a specified number of times.

$${q}_{i , m}^{out} = {q}_{i , 0} + {\sum }_{j = 1 , j \neq m}^{{w}_{i}} {q}_{i , j}^{i n} , \\ m = 1 , 2 , … , {w}_{i} , i = 1 , 2 , … , n \qquad ( 4 )$$

Herein, the second log-likelihood ratio calculating unit 4312 does not take the first log-likelihood ratio t_{j,m′} transmitted from the check node cⱼ being a destination to the variable node vᵢ being an originate into consideration when updating the second log-likelihood ratio q_{i,m} transmitted from one variable node vᵢ to one check node cⱼ along the edge as represented by equation (4). For example, when updating a second log-likelihood ratio q_{1,1} transmitted from the first variable node v₁ to the first check node c₁ along the edge, a first log-likelihood ratio t_{1,1} transmitted from the first check node c₁ to the first variable node v₁ is not taken into consideration.

The first log-likelihood ratio calculating unit 4311 reads the syndrome C of the target pixel included in the target line in the target frame from the memory unit 42 and calculates a first log-likelihood ratio t_{j,0} serving as an initial value of the first log-likelihood ratio t_{j,m′} based on the read syndrome C and standard deviation of noise in a communication channel. Then, the first log-likelihood ratio calculating unit 4311 transmits the calculated first log-likelihood ratio t_{j,0} from the check node cⱼ to the variable node vᵢ along the m′-th edge in the first likelihood change.

The first log-likelihood ratio calculating unit 4311 updates the first log-likelihood ratio t_{j,m′} by following equation (5) during the likelihood change between the first and second log-likelihood ratios performed a specified number of times.

$${t}_{j , {m}^{′}}^{out} = 2 {tanh}^{- 1} \{ ( 1 - 2 {s}_{j} ) {\prod }_{i = 1 , i \neq {m}^{′}}^{{r}_{j}} tanh ( \frac{{t}_{j , i}^{i n}}{2} ) \} , \\ {m}^{′} = 1 , 2 , … , {r}_{j} , j = 1 , 2 , … , n - k \qquad ( 5 )$$

In equation (5), sⱼ represents a value of a j-th bit of the read syndrome C.

Herein, the first log-likelihood ratio calculating unit 4311 does not take the second log-likelihood ratio q_{i,m} transmitted from the variable node vⱼ being the destination to the check node cⱼ being the originate into consideration when updating the first log-likelihood ratio t_{j,m′} transmitted from one check node cⱼ to one variable node vᵢ along the edge as represented by equation (5). For example, when calculating the first log-likelihood ratio t_{1,1} transmitted from the first check node c₁ to the first variable node v₁ along the edge, the second log-likelihood ratio q_{1,1} transmitted from the first variable node v₁ to the first check node c₁ is not taken into consideration.

The estimating unit 4313 estimates the Gray code (bit sequence) corresponding to the pixel value of the target pixel in the target line (non-key line) before the coding process by following equation (6) after the likelihood change between the first and second log-likelihood ratios is performed a specified number of times between the variable node vᵢ and the check node cⱼ (after repeated decoding).

$${\hat{x}}_{i} = \{ \begin{matrix}0 , & if {q}_{i , 0} + {\sum }_{m = 1}^{{w}_{i}} {q}_{i , m}^{i n} \geq 0 \\ 1 , & if {q}_{i , 0} + {\sum }_{m = 1}^{{w}_{i}} {q}_{i , m}^{i n} < 0\end{matrix} \qquad ( 6 )$$

In equation (6), xᵢ with a hat symbol represents the Gray code (bit sequence) corresponding to the pixel value of the target pixel in the target line (non-key line) estimated by the estimating unit 4313.

That is to say, the estimating unit 4313 adds the second log-likelihood ratio q_{i,0} serving as the initial value to all the first log-likelihood ratios t_{j,m′} transmitted to the variable node vᵢ through respective edges and estimates whether the value of an i-th bit of the pixel value of the target pixel in the target line (non-key line) is “0” or “1” by the value of the added log-likelihood ratios (posteriori log-likelihood ratio of the non-key line restored by the repeated decoding) as represented by equation (6).

The error detecting unit 432 performs parity check on the non-key line (target line) estimated by the decoding process by the decoding unit 431 to detect whether there is an error. Meanwhile, in the parity check, the low-density parity check matrix H used in the imaging device 3 (coding unit 321) is used.

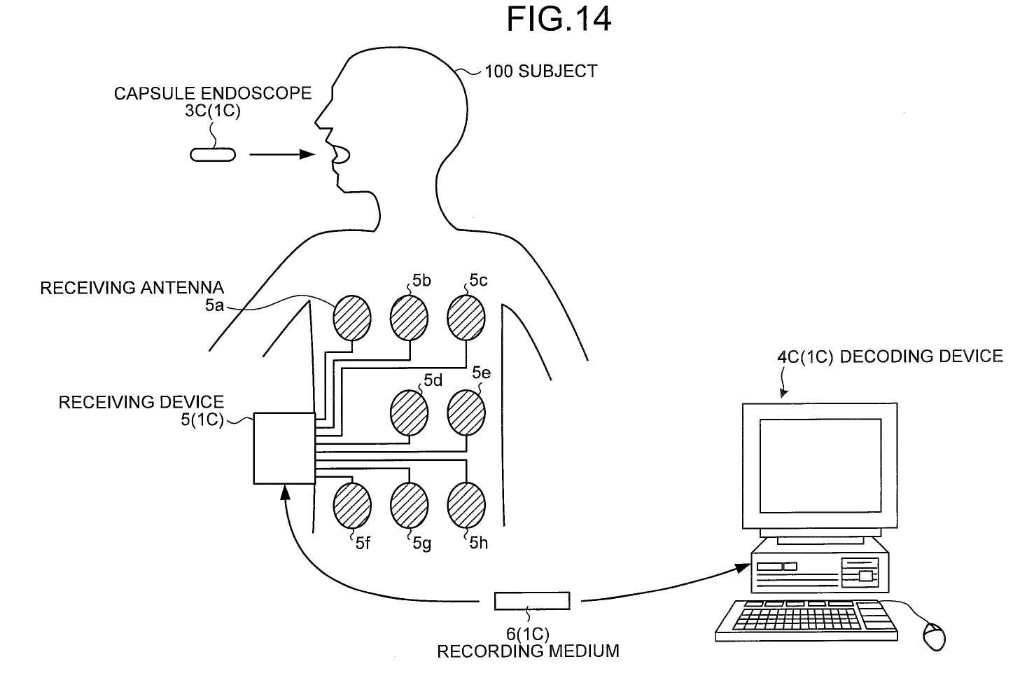

The display determining unit 433 performs a process of determining whether the frame (target frame) including the non-key line (target line) estimated by the decoding process by the decoding unit 431 is made a display target to be displayed on a display unit (for example, display unit 46 and the like illustrated in FIG. 15) based on a detection result by the error detecting unit 432. By the determining process, the display determining unit 433 adds a non-display target flag indicating a non-display target to the target frame when this is determined not to be the display target. When the moving image after being decoded by the decoding device 4 is displayed, the image corresponding to the frame to which the non-display target flag is not added is displayed on the display unit. On the other hand, the image corresponding to the frame to which the non-display target flag is added is not displayed on the display unit.

The synthesizing unit 434 reconstructs the image data of one frame from the non-key line estimated by the decoding process by the decoding unit 431 and the key line stored in the memory unit 42 which forms the same frame as the non-key line. Then, the synthesizing unit 434 creates the moving image file in which a plurality of reconstructed image data is arranged in chronological order.

The Gray decoding unit 435 performs Gray decoding on the moving image file generated by the synthesizing unit 434 (converts the Gray code to the pixel value).

Meanwhile, when the control unit 43 detects an error in a decoded result of the non-key line in the repeated decoding in a forward direction, this may shift to the repeated decoding in a trace back direction. Herein, the repeated decoding in the forward direction is intended to mean the repeated decoding performed by using the second log-likelihood ratio q_{i,0} serving as the initial value calculated by using the key line “which comes immediately before” the target line (non-key line) in chronological order in the target frame. The repeated decoding in the trace back direction is intended to mean the repeated decoding performed by using the second log-likelihood ratio q_{i,0} serving as the initial value calculated by using the key line which comes immediately after the target line (non-key line) in chronological order in the target frame (with a larger row number adjacently located in the target frame). When all the non-key lines which cannot be correctly decoded in the repeated decoding in the forward direction are correctly decoded in the repeated decoding in the trace back direction, the control unit 43 finishes the decoding process. On the other hand, when there remains the non-key line which cannot be correctly decoded, the control unit 43 shifts to a decoding mode in which linear interpolation is utilized.

In the decoding mode in which the linear interpolation is utilized, the control unit 43 performs the repeated decoding by calculating a predicted luminance value of each pixel of the non-key line which cannot be decoded by the linear interpolation by using the correctly decoded non-key line or key line and giving the log-likelihood ratio based on the predicted value as the second log-likelihood ratio.

Operation of Imaging System

Next, operation (coding/decoding method) of the above-described imaging system 1 is described.

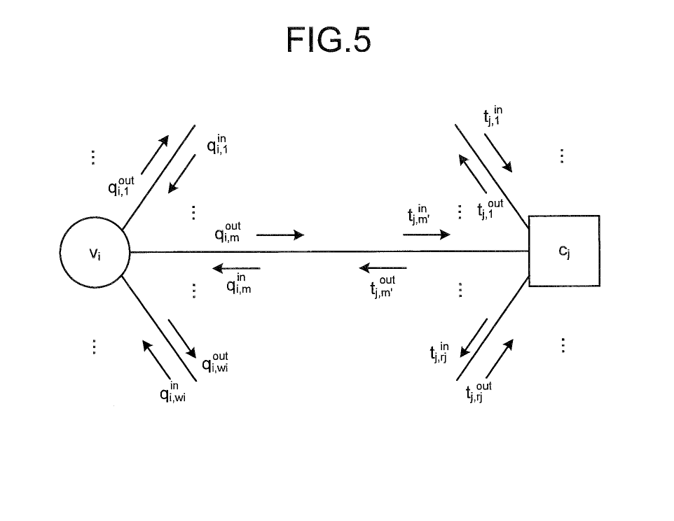

FIG. 6 is a flowchart illustrating the coding/decoding method according to the first embodiment of the present invention.

Meanwhile, it is hereinafter described in order of operation of the imaging device 3 and operation of the decoding device 4 for the purpose of description.

Operation of Imaging Device

First, the imaging element 312 starts capturing an image of the subject (at a frame rate of 30 frames per second, for example) under control of the control unit 32 (step S1).

After step S1, the sorting unit 315 sorts the moving image frame sequence captured by the imaging element 312 to be Gray coded through the signal processing unit 313 and the Gray coding unit 314 into the key line and the non-key line for each frame, and outputs the key line and the non-key line to the transmitting unit 33 and the coding unit 321, respectively (step S2: sorting step).

After step S2, the coding unit 321 to which the non-key line from the sorting unit 315 is input performs the coding process (syndrome coding) on the non-key line (step S3: coding step).

After step S3, the transmitting unit 33 makes the key line from the sorting unit 315 and the non-key line after the coding process (syndrome C) from the coding unit 321 the data stream under control of the control unit 32. The transmitting unit 33 transmits the moving image data which is made the data stream to the decoding device 4 through the wireless transmission system 2 (step S4: transmitting step).

Operation of Decoding Device (Decoding Method)

The control unit 43 reads the decoding program from the memory unit 42 and executes the following process according to the decoding program.

First, the receiving unit 41 sequentially receives the moving image data from the imaging device 3 to output to the memory unit 42 under control of the control unit 43 (step S5: receiving step and data obtaining step). The memory unit 42 sequentially stores the received data.

After step S5, the decoding unit 431 performs the decoding process in block to all the pixels included in the non-key line as described below (step S6: decoding step).

Hereinafter, in order to specifically describe the decoding process (step S6), the description focuses on one pixel included in the non-key line.

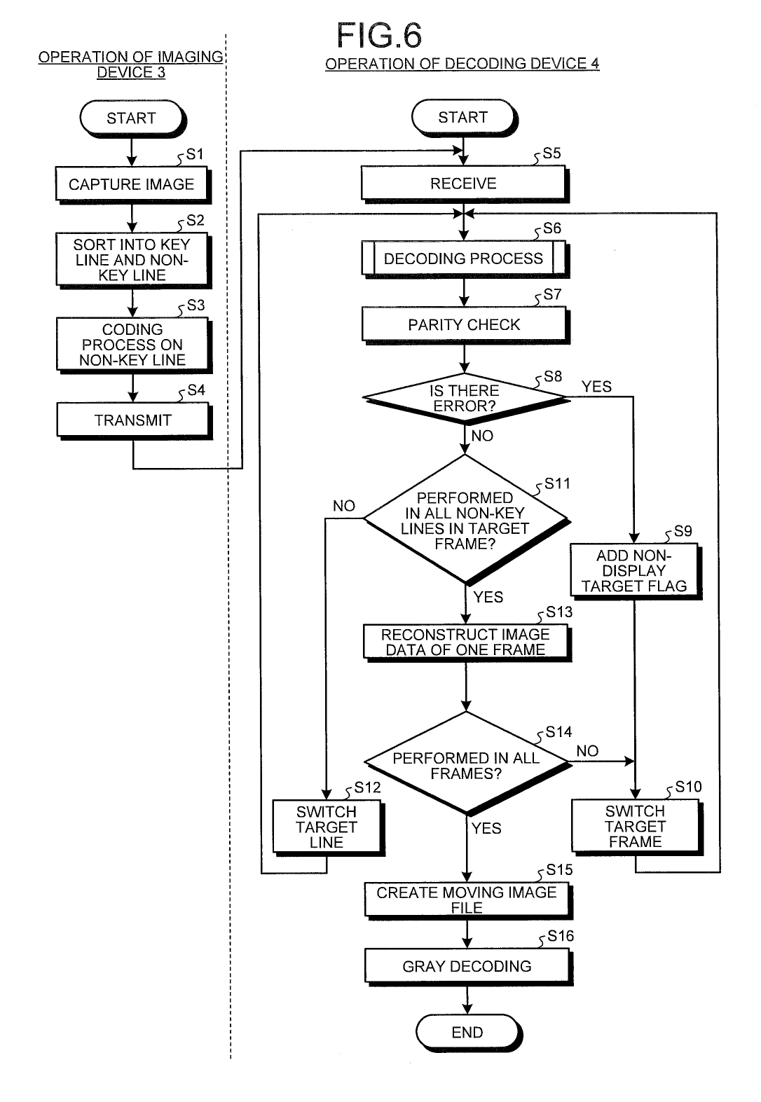

FIG. 7 is a flowchart illustrating the decoding process according to the first embodiment of the present invention.

First, the second log-likelihood ratio calculating unit 4312 reads the key line immediately before the target line in chronological order in the target frame and the characteristic information corresponding to the target pixel included in the target line from the memory unit 42 and calculates the second log-likelihood ratio q_{i,0} serving as the initial value (step S61).

After step S61, the first log-likelihood ratio calculating unit 4311 reads the target line (syndrome C of the target pixel) in the target frame from the memory unit 42 and calculates the first log-likelihood ratio t_{j,0} serving as the initial value based on the read syndrome C (step S62).

After step S62, the decoding unit 431 performs the likelihood change between the first and second log-likelihood ratios a specified number of times. The first and second log-likelihood ratio calculating units 4311 and 4312 update the first and second log-likelihood ratios t_{j,m′} and q_{i,m} by equations (5) and (4), respectively, during the likelihood change (step S63).

After step S63, the estimating unit 4313 estimates the non-key line before the coding process (Gray code (bit sequence) of the target pixel) using equation (6) based on the posteriori log-likelihood ratio of the non-key line restored by the repeated decoding (step S63) (step S64).

Then, the decoding unit 431 performs the above-described process (steps S61 to S64) in block to all the pixels included in the target line (non-key line) and finishes the decoding process (step S6).

After step S6, the error detecting unit 432 performs the parity check on the non-key line estimated by the decoding unit 431 (step S7) to determine whether there is an error (step S8).

When it is determined “Yes” at step S8, that is to say, when it is determined that there is an error by the parity check, the display determining unit 433 adds the non-display target flag to the target frame (step S9).

After step S9, the control unit 43 switches the target frame to a next frame (step S10), shifts to step S6, and performs the decoding process on the non-key line in the switched target frame.

On the other hand, when it is determined “No” at step S8, that is to say, it is determined that there is no error by the parity check, the control unit 43 determines whether step S6 is performed on all the non-key lines in the target frame (step S11).

When it is determined “No” at step S11, the control unit 43 switches the target line in the target frame to a next non-key line (step S12), shifts to step S6, and performs the decoding process on the switched target line.

When it is determined “Yes” at step S11, the synthesizing unit 434 reconstructs the image data of one frame from the non-key line after the decoding process by the decoding unit 431 (step S6) and the key line stored in the memory unit 42 which forms the same frame as the non-key line (step S13).

After step S13, the control unit 43 determines whether step S6 is performed on all the frames stored in the memory unit 42 (step S14).

When it is determined “No” at step S14, the control unit 43 switches the target frame to a next frame (step S10), shifts to step S6, and performs the decoding process on the non-key line in the switched target frame.

On the other hand, when it is determined “Yes” at step S14, the synthesizing unit 434 creates the moving image file in which a plurality of image data reconstructed at step S13 is arranged in chronological order (step S15).

Then, the Gray decoding unit 435 performs the Gray decoding on the moving image file generated at step S15 (step S16).

In the first embodiment of the present invention described above, the imaging device 3 does not code the key line but performs the coding process on the non-key line out of the moving image data generated by capturing. Then, the imaging device 3 transmits the key line and the non-key line as the data stream. Therefore, it is possible to decrease an information amount of the moving image data to be transmitted. It is also possible to make a data length of the moving image data to be transmitted identical. Furthermore, secrecy of the moving image data may be improved by the coding process.

The decoding device 4 performs the repeated decoding by the belief-propagation method based on the first log-likelihood ratio t_{j,0} serving as the initial value obtained from the non-key line after the coding process and the second log-likelihood ratio q_{i,0} serving as the initial value obtained from the key-line which is not coded and the characteristic information. Therefore, it is possible to adopt a simple coding system as the coding system performed by the imaging device 3. Specifically, in the first embodiment, the syndrome coding using the low-density parity check matrix H is adopted as the coding process, it is only required to apply the low-density parity check matrix H to the non-key line, so that a calculation amount of the coding process is very small.

From above, it is possible to realize the imaging system 1, the decoding device 4, the coding/decoding method, the decoding method, and the decoding program capable of inhibiting a load and consumed power of the imaging device 3 even when the frame rate at the time of imaging is made large (for example, at a frame rate of 30 frames per second).

Furthermore, since the decoding device 4 performs the repeated decoding, this might correct an error occurring in association with transmission/reception and storage of the moving image data.

Correlation in the frame (correlation between the key line and the non-key line) is higher than that between the frames as the pixel value correlation characteristic.

The decoding device 4 calculates the second log-likelihood ratio q_{i,0} serving as the initial value using the key line which comes immediately before the target line (non-key line) in chronological order, that is to say, the key line having high correlation with the target line and the characteristic information and performs the repeated decoding by using the second log-likelihood ratio q_{i,0}. Therefore, it is possible to estimate the non-key line before the coding process with a high degree of accuracy.

Regarding the pixel value correlation characteristic, the correlation between the pixels of the same type of corresponding pixel groups (for example, red pixel and red pixel) is higher than the correlation between those of different types of corresponding pixel groups (for example, red pixel and blue pixel) in one frame.

The decoding device 4 uses the characteristic information corresponding to the type of the target pixel (for example, the characteristic information for red pixel when the target pixel is the red pixel) when calculating the second log-likelihood ratio q_{i,0} serving as the initial value. Therefore, it is possible to estimate a luminance value of each pixel included in the non-key line before the coding process with a higher degree of accuracy.

The decoding device 4 detects an error by the parity check after correcting an error by the repeated decoding (estimating the non-key line with a high degree of accuracy), and adds the non-display target flag to the target flag including the non-key line in which the error is detected and does not make the same the display target. Therefore, it is possible to realize display in which deterioration in image quality from the moving image data generated by the imaging device 3 is inhibited when playing back the moving image file.

### Second Embodiment

A second embodiment of the present invention is next described.

In the following description, the same reference sign is assigned to a configuration and a step similar to those of the above-described first embodiment and detailed description thereof is omitted or simplified.

A memory unit 42 stores a piece of characteristic information for each of red pixel, blue pixel, first green pixel, and second green pixel as characteristic information in the above-described first embodiment. When performing a decoding process in block to all pixels included in a non-key line, a decoding unit 431 calculates a second log-likelihood ratio q_{i,0} serving as an initial value by using the characteristic information corresponding to a type of a target pixel for each of the types of the target pixels (four types of red pixel, blue pixel, first green pixel, and second green pixel) and performs repeated decoding by using the second log-likelihood ratio q_{i,0} (step S63).

On the other hand, in the second embodiment, the memory unit 42 stores a plurality of pieces of characteristic information for each corresponding pixel group (characteristic information for each of red pixel, blue pixel, first green pixel, and second green pixel).

That is to say, a plurality of pieces of characteristic information for each corresponding pixel group (a plurality of pieces of characteristic information for red pixel, a plurality of pieces of characteristic information for blue pixel, a plurality of pieces of characteristic information for first green pixel, and a plurality of pieces of characteristic information for second green pixel) is calculated from a plurality of image data with different time periods and places at which they are captured. Therefore, the plurality of pieces of characteristic information has different probability distributions as illustrated in FIG. 4B.

When performing the decoding process in block to all the pixels included in the non-key line, the decoding unit 431 changes the second log-likelihood ratio q_{i,0} serving as the initial value by using a plurality of pieces of characteristic information corresponding to the type of the target pixel for each type of the target pixel when a specified condition is satisfied and performs repeated decoding by using the changed second log-likelihood ratio q_{i,0} as described below.

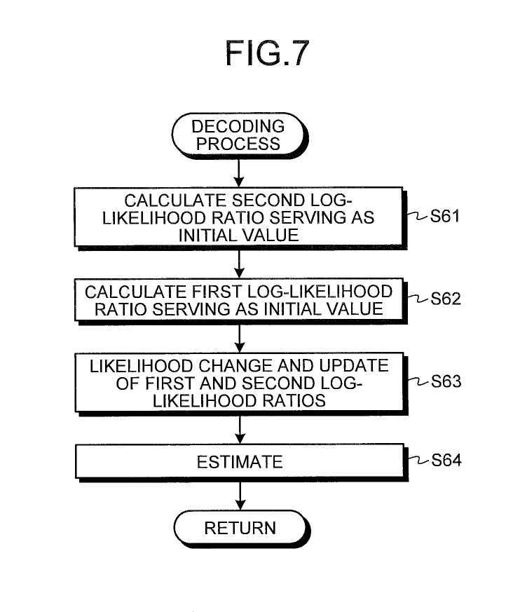

FIG. 8 is a flowchart illustrating a coding/decoding method according to the second embodiment of the present invention.

In the coding/decoding method according to the second embodiment, operation of an imaging device 3 is similar to that of the above-described first embodiment. Therefore, in FIG. 8, the operation of the imaging device 3 is not illustrated and only operation of a decoding device 4 (decoding method) is illustrated.

The decoding method according to the second embodiment is different from the decoding method described in the above-described first embodiment only in that following steps S17 and S18 are added.

Therefore, only steps S17 and S18 are hereinafter described.

Step S17 is performed when it is determined “Yes” at step S8 as a result of parity check (step S7), that is to say, when it is determined that there is an error (corresponding to a case in which the above-described specified condition is satisfied).

At step S17, a control unit 43 determines whether all pieces of characteristic information (all of a plurality of pieces of characteristic information for each corresponding pixel group) used for calculating the second log-likelihood ratio q_{i,0} serving as the initial value stored in a memory unit 42 are used.

When it is determined “No” at step S17, the control unit 43 (second log-likelihood ratio calculating unit 4312) calculates the second log-likelihood ratio q_{i,0} serving as the initial value as at step S61 by using characteristic information different from the characteristic information previously used out of a plurality of pieces of characteristic information corresponding to the type of the target pixel (a plurality of pieces of characteristic information for red pixel when the target pixel is the red pixel) for each type of the target pixel and changes the second log-likelihood ratio q_{i,0} previously used to the calculated second log-likelihood ratio q_{i,0} (step S18).

After step S18, the decoding unit 431 shifts to step S63 and performs new likelihood change by using the second log-likelihood ratio q_{i,0} serving as the initial value changed at step S18 and a first log-likelihood ratio t_{j,0} serving as an initial value calculated at step S62 for each type of the target pixel.

On the other hand, when it is determined “Yes” at step S17, that is to say, when it is determined that all the pieces of characteristic information used for calculating the second log-likelihood ratio q_{i,0} serving as the initial value are used, the control unit 43 shifts to step S9 and adds a non-display target flag to a target frame.

The second embodiment of the present invention described above has the following effect in addition to the effect similar to that of the above-described first embodiment.

In the second embodiment, when performing the decoding process in block to all the pixels included in the non-key line, the decoding unit 431 changes the second log-likelihood ratio q_{i,0} serving as the initial value by using a plurality of pieces of characteristic information corresponding to the type of the target pixel for each of the types of the target pixels and performs the repeated decoding by using the changed second log-likelihood ratio q_{i,0}. Therefore, it is possible to estimate the non-key line with a higher degree of accuracy.

Meanwhile, although the decoding device 4 changes the second log-likelihood ratio q_{i,0} serving as the initial value only when an error is detected as a result of the parity check (step S7) (step S18) in the above-described second embodiment, there is no limitation. For example, it may also be configured such that the second log-likelihood ratio q_{i,0} serving as the initial value is calculated by using each of all the pieces of characteristic information corresponding to the type of the target pixel for each type of the target pixel and the repeated decoding is performed by using all the second log-likelihood ratios q_{i,0}. At that time, the decoding device 4 may create a moving image file by using the non-key line determined to have no error by the parity check out of the non key-lines estimated after each repeated decoding.

### Third Embodiment

A third embodiment of the present invention is next described.

In the following description, the same reference sign is assigned to a configuration and a step similar to those of the above-described first embodiment and detailed description thereof is omitted or simplified.

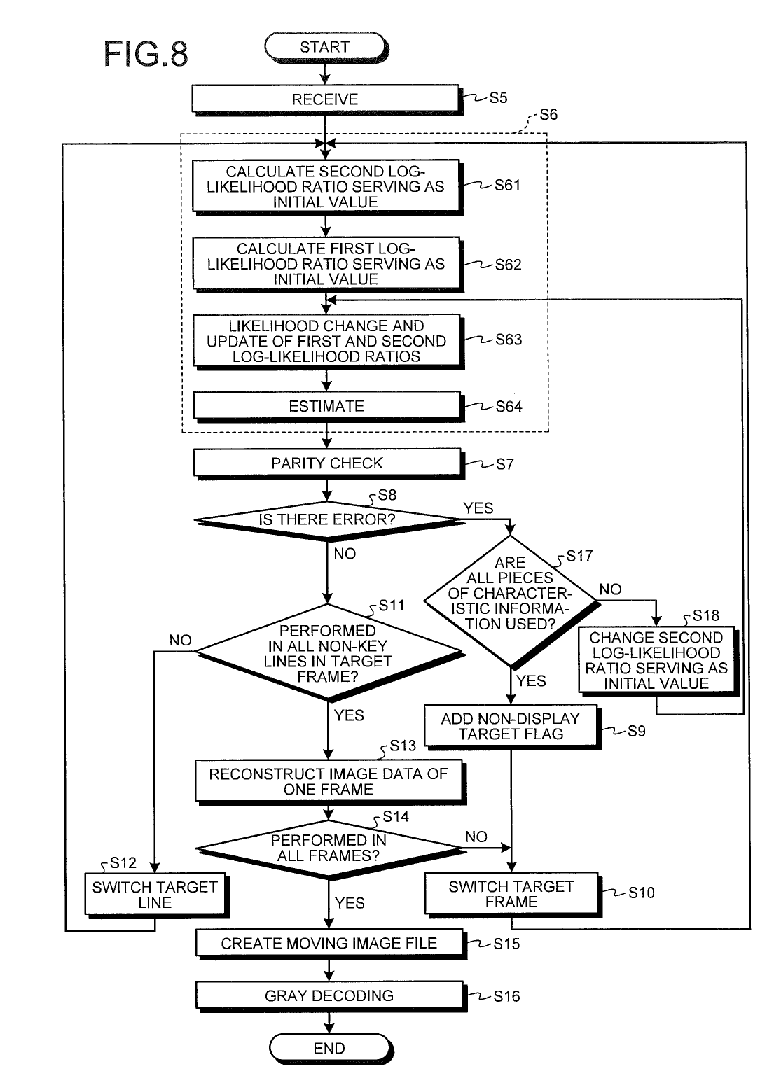

FIG. 9 is a block diagram illustrating an imaging system 1A according to the third embodiment of the present invention.

In the above-described first embodiment, a display determining unit 433 performs a process of determining whether a target frame is made a display target based on a result of parity check (step S7).

On the other hand, the imaging system 1A according to the third embodiment is provided with a decoding device 4A (control unit 43A) obtained by removing an error detecting unit 432 and adding a display determining unit 433A obtained by changing a part of function of the display determining unit 433 as illustrated in FIG. 9 as compared to an imaging system 1 (FIG. 1) described in the above-described first embodiment.

The display determining unit 433A performs a determining process based on a posteriori log-likelihood ratio of a non-key line restored by repeated decoding by a decoding unit 431 as described below.

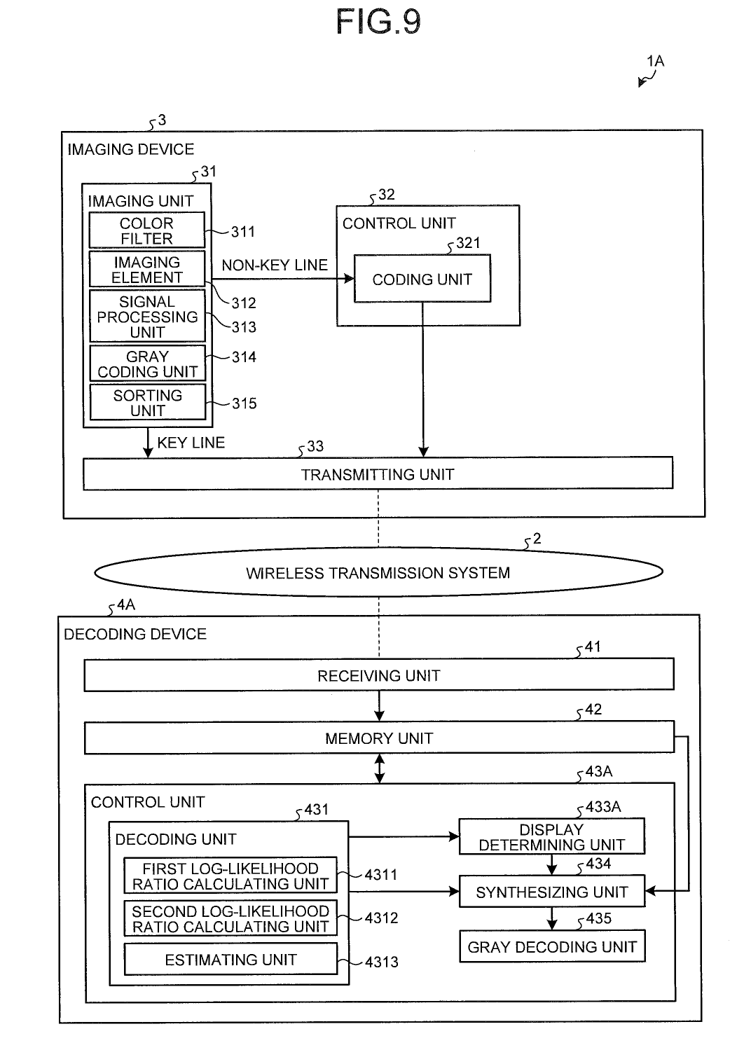

FIG. 10 is a flowchart illustrating a coding/decoding method according to the third embodiment of the present invention.

In the coding/decoding method according to the third embodiment, operation of an imaging device 3 is similar to that of the above-described first embodiment.

The decoding method according to the third embodiment is different from the decoding method described in the above-described first embodiment only in that steps S19 and S20 are added in place of steps S7 and S8.

Therefore, only steps S19 and S20 are hereinafter described.

Step S19 is performed after a decoding process (step S6).

At step S19, the display determining unit 433A compares an absolute value of the posteriori log-likelihood ratio of the non-key line restored by the repeated decoding at step S6 with a first threshold for each bit of a Gray code (bit sequence) for all pixels included in a target line.

After step S19, the display determining unit 433A determines whether the number of bits with which the absolute value of the posteriori log-likelihood ratio is smaller than the first threshold is larger than a second threshold (step S20).

When it is determined “Yes” at step S20, the display determining unit 433A shifts to step S9 and adds a non-display target flag to the target frame.

On the other hand, when it is determined “No” at step S20, the control unit 43A shifts to step S11.

Also in a case in which the determining process by the display determining unit 433A is performed based on the posteriori log-likelihood ratio of the non-key line restored by the repeated decoding as in the third embodiment of the present invention described above, an effect similar to that of the above-described first embodiment is obtained.

Meanwhile, although a configuration in which the determining process based on the posteriori log-likelihood ratio of the non-key line restored by the repeated decoding is applied to the above-described first embodiment is described in the above-described third embodiment, there is no limitation, and this may also be applied to the above-described second embodiment.

Although the target frame is made a non-display target when the number of bits with which the absolute value of the posteriori log-likelihood ratio is smaller than the first threshold is larger than the second threshold in the above-described third embodiment, there is no limitation, and another method may also be adopted when the determining process is performed based on the posteriori log-likelihood ratio.

For example, a bit level of the Gray code (bit sequence) is weighted (lower bit is weighted heavier, for example). A product of the weight and the absolute value of the posteriori log-likelihood ratio is obtained for each bit of the Gray code for all the pixels included in the target line, and when a sum of them is smaller than a third threshold, the target frame is made the non-display target.

### Fourth Embodiment

A fourth embodiment of the present invention is next described.

In the following description, the same reference sign is assigned to a configuration and a step similar to those of the above-described first embodiment and detailed description thereof is omitted or simplified.

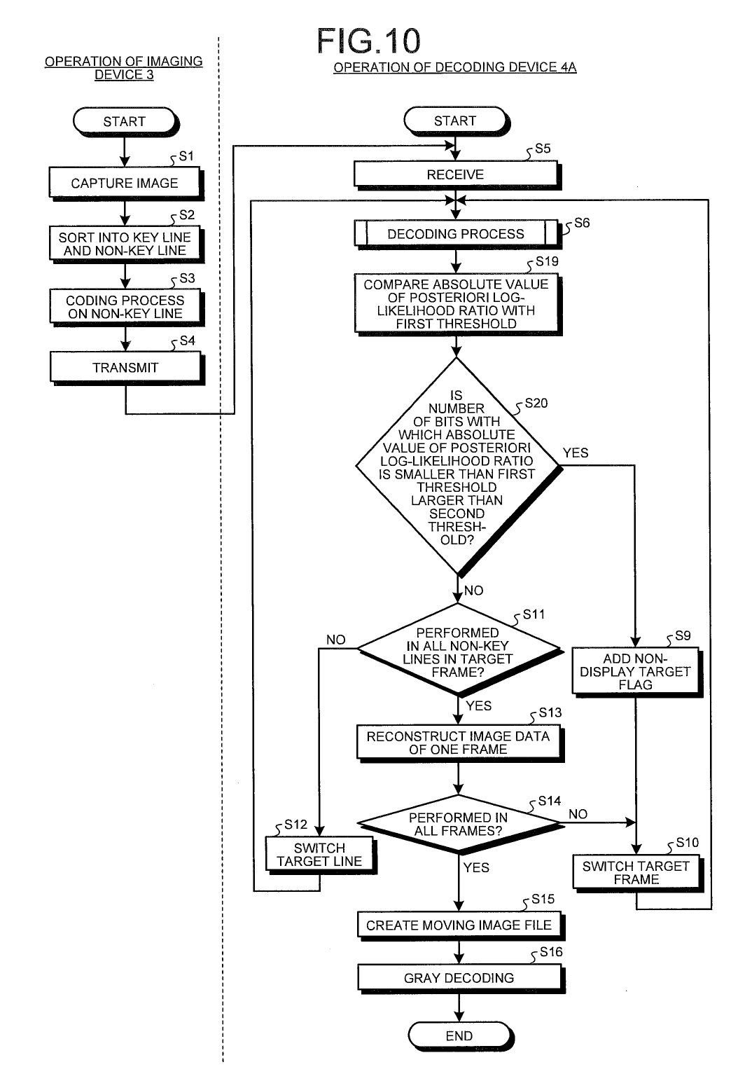

FIG. 11 is a block diagram illustrating an imaging system 1B according to the fourth embodiment of the present invention.

The imaging system 1B according to the fourth embodiment performs a coding process and a decoding process for each type of a corresponding pixel group as compared to an imaging system 1 (FIG. 1) described in the above-described first embodiment.

In an imaging device 3B which forms the imaging system 1B according to the fourth embodiment, a part of a function of a sorting unit 315 is changed as illustrated in FIG. 11 as compared to an imaging device 3 described in the above-described first embodiment.

A sorting unit 315B according to the fourth embodiment first sorts image data (moving image frame sequence) Gray coded by a Gray coding unit 314 according to the types of the corresponding pixel groups for each frame.

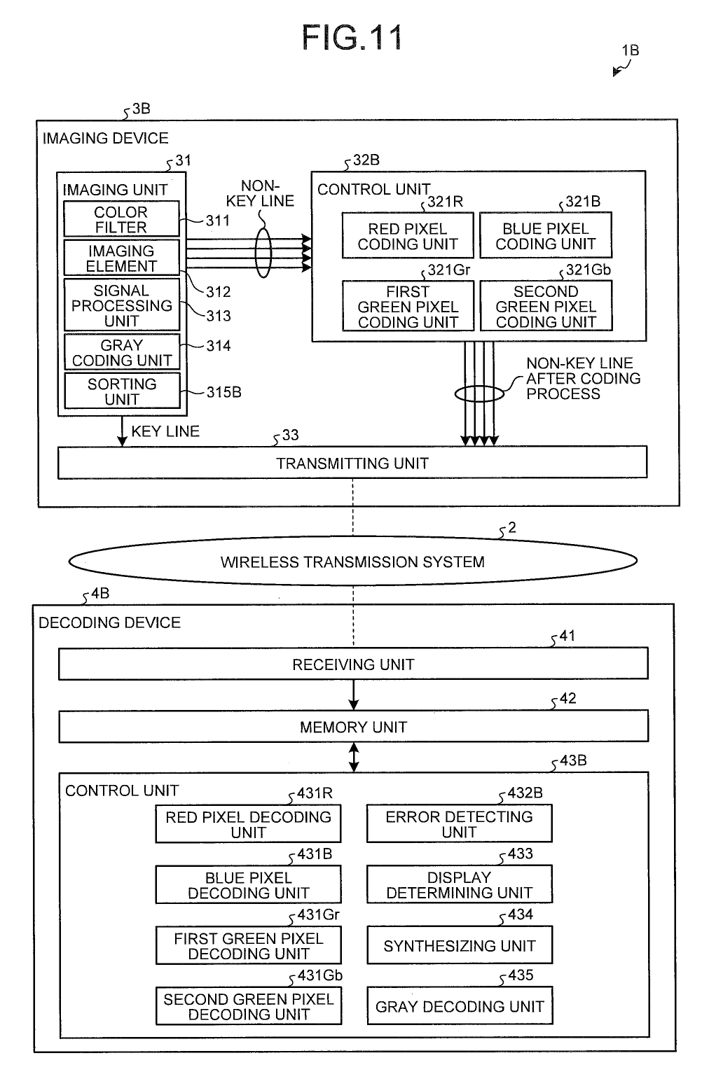

FIG. 12 is a view virtually illustrating the function of the sorting unit 315B according to the fourth embodiment of the present invention. Meanwhile, in FIG. 12, as in FIG. 2, a sign “R” is assigned to each red pixel, a sign “B” is assigned to each blue pixel, a sign “Gr” is assigned to each first green pixel, and a sign “Gb” is assigned to each second green pixel in an image F of one frame.

Specifically, the sorting unit 315B sorts the image F into a red pixel sub frame FR, a blue pixel sub frame FB, a first green pixel sub frame FGr, and a second green pixel sub frame FGb according to the types of the corresponding pixel group as illustrated in FIG. 12.

The red pixel sub frame FR is the frame in which the red pixels R arranged in a first row of the image F are sequentially arranged in a first row from a first column in ascending order of column numbers, the red pixels R arranged in a third row of the image F are sequentially arranged in a second row from the first column in ascending order of column numbers, and the pixels are arranged in third and subsequent rows in the same manner.

The blue pixel sub frame FB is the frame in which the blue pixels B arranged in a second row of the image F are sequentially arranged in a first row from a first column in ascending order of column numbers, the blue pixels B arranged in a fourth row of the image F are sequentially arranged in a second row from the first column in ascending order of column numbers, and the pixels are arranged in third and subsequent rows in the same manner.

The first green pixel sub frame FGr is the frame in which the first green pixels Gr arranged in the second row of the image F are sequentially arranged in a first row from a first column in ascending order of column numbers, the first green pixels Gr arranged in the fourth row of the image F are sequentially arranged in a second row from the first column in ascending order of column numbers, and the pixels are arranged in third and subsequent rows in the same manner.

The second green pixel sub frame FGb is the frame in which the second green pixels Gb arranged in the first row of the image F are sequentially arranged in a first row from the first column in ascending order of column numbers, the second green pixels Gb arranged in the third row of the image F are sequentially arranged in a second row from the first column in ascending order of column numbers, and the pixels are arranged in third and subsequent rows in the same manner.

Next, the sorting unit 315B makes a plurality of pixels of one row out of a plurality of pixels arranged in a matrix pattern one block for each sub frame and sorts such that one out of several blocks is made a key line and the others are made non-key lines in ascending order of row numbers as illustrated in FIG. 12. In an example in FIG. 12, the sorting unit 315B makes one out of four blocks the key line.

In the fourth embodiment, the number of key lines is the same in the red pixel sub frame Fr, the blue pixel sub frame FB, the first green pixel sub frame FGr, and the second green pixel sub frame FGb. The number of non-key lines is also the same.

Hereinafter, for the purpose of description, the key line and the non-key line sorted from the red pixel sub frame FR by the sorting unit 315B are referred to as a red pixel key line and a red pixel non-key line, respectively. Similarly, the key line and the non-key line sorted from the blue pixel sub frame FB, the key line and the non-key line sorted from the first green pixel sub frame FGr, and the key line and the non-key line sorted from the second green pixel sub frame FG are referred to as a blue pixel key line and a blue pixel non-key line, a first green pixel key line and a first green pixel non-key line, and a second green pixel key line and a second green pixel non-key line, respectively.

The sorting unit 315B outputs the key lines to a transmitting unit 33 and outputs the red pixel non-key line, the blue pixel non-key line, the first green pixel non-key line, and the second green pixel non-key line to a control unit 32B.

The imaging device 3B according to the fourth embodiment is provided with four coding units 321 (red pixel coding unit 321R, blue pixel coding unit 321B, first green pixel coding unit 321Gr, and second green pixel coding unit 321Gb) corresponding to the types of the corresponding pixel groups as illustrated in FIG. 11 as compared to the imaging device 3 (FIG. 1) described in the above-described first embodiment.

Specifically, the red pixel coding unit 321R to which the red pixel non-key line from the sorting unit 315B is sequentially input performs syndrome coding as the coding unit 321 described in the above-described first embodiment for each red pixel non-key line.

The blue pixel coding unit 321B, the first green pixel coding unit 321Gr, and the second green pixel coding unit 321Gb to which the corresponding blue pixel non-key line, first green pixel non-key line, and second green pixel non-key line are sequentially input from the sorting unit 315B, respectively, perform the syndrome coding as the coding unit 321 described in the above-described first embodiment for each non-key line.

Herein, in at least one coding unit out of the coding units 321R, 321B, 321Gr, and 321Gb, a low-density parity check matrix used in the syndrome coding is different from the low-density parity check matrix used in the syndrome coding in other coding units.

A decoding device 4B forming the imaging system 1B according to the fourth embodiment is provided with four decoding units 431 (red pixel decoding unit 431R, blue pixel decoding unit 431B, first green pixel decoding unit 431Gr, and second green pixel decoding unit 431Gb) corresponding to the types of the corresponding pixel groups as illustrated in FIG. 11 as compared to a decoding device 4 (FIG. 1) described in the above-described first embodiment.

Each of the red pixel decoding unit 431R, the blue pixel decoding unit 431B, the first green pixel decoding unit 431Gr, and the second green pixel decoding unit 431Gb is provided with a first log-likelihood ratio calculating unit 4311, a second log-likelihood ratio calculating unit 4312, and an estimating unit 4313 as the decoding unit 431 described in the above-described first embodiment. Meanwhile, configurations thereof are not illustrated in FIG. 11.

The red pixel decoding unit 431R, the blue pixel decoding unit 431B, the first green pixel decoding unit 431Gr, and the second green pixel decoding unit 431Gb perform the decoding process similar to that described in the above-described first embodiment to estimate the red pixel non-key line, the blue pixel non-key line, the first green pixel non-key line, and the second green pixel non-key line, respectively, before the coding process by the imaging device 3B.

Herein, the red pixel decoding unit 431R, the blue pixel decoding unit 431B, the first green pixel decoding unit 431Gr, and the second green pixel decoding unit 431Gb are different from the decoding unit 431 described in the above-described first embodiment in information used for calculating first and second log-likelihood ratios t_{j,0} and q_{i,0} serving as initial values.

Specifically, the red pixel decoding unit 431R uses the following information stored in a memory unit 42 when calculating the first and second log-likelihood ratios t_{j,0} and q_{i,0} serving as the initial values.

That is to say, when calculating the second log-likelihood ratio q_{i,0} serving as the initial value, the red pixel decoding unit 431R uses the red pixel key line which comes immediately before a target line (red pixel non-target line) in chronological order (with a smaller row number adjacently located in the red pixel frame) in the red pixel sub frame forming a target frame and the characteristic information for red pixel.

The red pixel decoding unit 431R uses a syndrome C of a target pixel included in the target line (red pixel non-key line) in the red pixel sub frame forming the target frame and standard deviation of noise in a communication channel when calculating the first log-likelihood ratio t_{j,0} serving as the initial value.

Then, the red pixel decoding unit 431R uses the first and second log-likelihood ratios t_{j,0} and q_{i,0} serving as the initial values calculated by using the above-described information in first likelihood change, then performs the likelihood change a specified number of times and estimates the red pixel non-key line before the coding process as in the above-described first embodiment.

The blue pixel decoding unit 431B uses the following information stored in the memory unit 42 when calculating the first and second log-likelihood ratios t_{j,0} and q_{i,0} serving as the initial values.

That is to say, the blue pixel decoding unit 431B uses the blue pixel key line which comes immediately before the target line (blue pixel non-key line) in chronological order (with a smaller row number adjacently located in the blue pixel frame) in the blue pixel sub frame forming the target frame and the characteristic information for blue pixel when calculating the second log-likelihood ratio q_{i,0} serving as the initial value.

The blue pixel decoding unit 431B uses the syndrome C of the target pixel included in the target line (blue pixel non-key line) in the blue pixel sub frame forming the target frame and the standard deviation of the noise in the communication channel when calculating the first log-likelihood ratio t_{j,0} serving as an initial value.

Then, the blue pixel decoding unit 431B uses the first and second log-likelihood ratios t_{j,0} and q_{i,0} serving as the initial values calculated by using the above-described information in the first likelihood change, then performs the likelihood change a specified number of times and estimates the blue pixel non-key line before the coding process as in the above-described first embodiment.

The first green pixel decoding unit 431Gr uses the following information stored in the memory unit 42 when calculating the first and second log-likelihood ratios t_{j,0} and q_{i,0} serving as the initial values.

That is to say, the first green pixel decoding unit 431Gr uses the first green pixel key line which comes immediately before the target line (first green pixel non-key line) in chronological order (with a smaller row number adjacently located in the first green pixel sub frame) in the first green pixel sub frame forming the target frame and the characteristic information for first green pixel when calculating the second log-likelihood ratio q_{i,0} serving as the initial value.

The first green pixel decoding unit 431Gr uses the syndrome C of the target pixel included in the target line (first green pixel non-key line) in the first green pixel sub frame forming the target frame and the standard deviation of the noise in the communication channel when calculating the first log-likelihood ratio t_{j,0} serving as the initial value.

Then, the first green pixel decoding unit 431Gr uses the first and second log-likelihood ratios t_{j,0} and q_{i,0} serving as the initial values calculated by using the above-described information in the first likelihood change, then performs the likelihood change a specified number of times and estimates the first green pixel non-key line before the coding process as in the above-described first embodiment.

The second green pixel decoding unit 431Gb uses the following information stored in the memory unit 42 when calculating the first and second log-likelihood ratios t_{j,0} and q_{i,0} serving as the initial values.

That is to say, the second green pixel decoding unit 431Gb uses the second green pixel key line which comes immediately before the target line (second green pixel non-key line) in chronological order (with a smaller row number adjacently located in the second green pixel sub frame) in the second green pixel sub frame forming the target frame and the characteristic information for second green pixel when calculating the second log-likelihood ratio q_{i,0} serving as the initial value.

The second green pixel decoding unit 431Gb uses the syndrome C of the target pixel included in the target line (second green pixel non-key line) in the second green pixel sub frame forming the target frame and the standard deviation of the noise in the communication channel when calculating the first log-likelihood ratio t_{j,0} serving as the initial value.

Then, the second green pixel decoding unit 431Gb uses the first and second log-likelihood ratios t_{j,0} and q_{i,0} serving as the initial values calculated by using the above-described information in the first likelihood change, then performs the likelihood change a specified number of times and estimates the second green pixel non-key line before the coding process as in the above-described first embodiment.

The decoding device 4B according to the fourth embodiment is obtained by changing a part of a function of an error detecting unit 432 of the decoding device 4 described in the above-described first embodiment as illustrated in FIG. 11.

An error detecting unit 432B according to the fourth embodiment performs parity check on the red pixel non-key line (target line) estimated by the decoding process by the red pixel decoding unit 431R, the blue pixel non-key line (target line) estimated by the decoding process by the blue pixel decoding unit 431B, the first green pixel non-key line (target line) estimated by the decoding process by the first green pixel decoding unit 431Gr, and the second green pixel non-key line (target line) estimated by the decoding process by the second green pixel decoding unit 431Gb to detect whether there is an error. Meanwhile, in the parity check on the red pixel non-key line, the low-density parity check matrix used by the red pixel coding unit 321R. In each parity check on the blue pixel non-key line, first green pixel non-key line, and second green pixel non-key line also, the low-density parity check matrix used by each of the coding units 321B, 321Gr, and 321Gb corresponding to each of the non-key lines is used in the above-described manner.

Next, operation of the above-described imaging system 1B (coding/decoding method) is described.

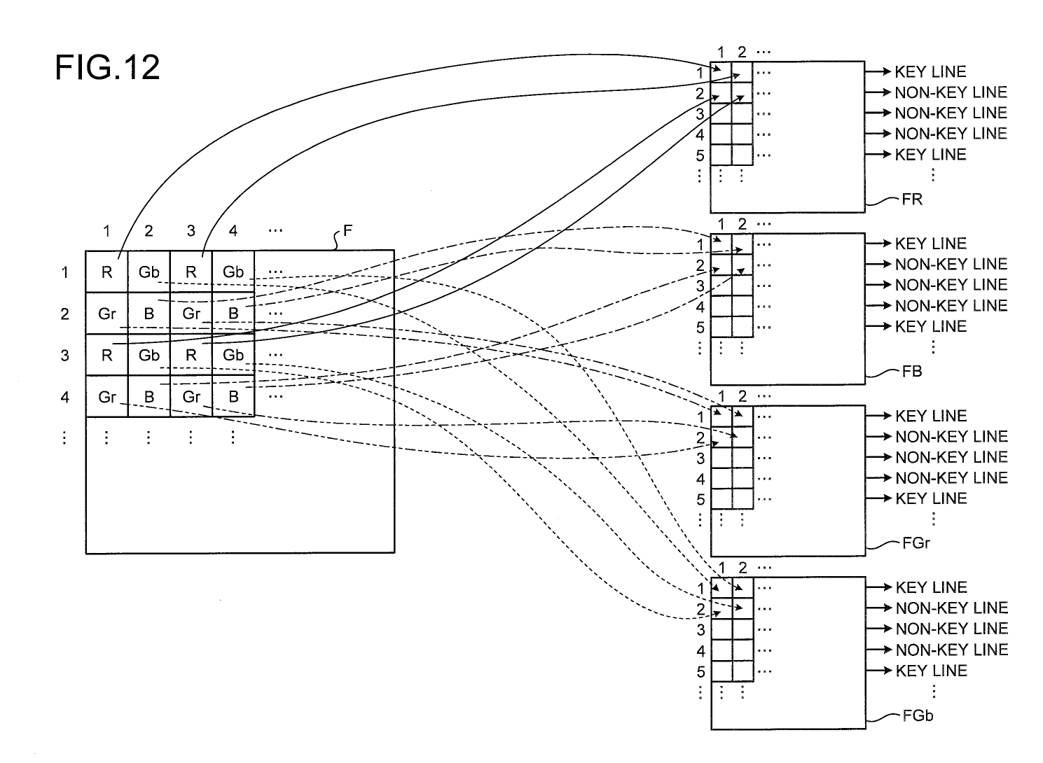

FIG. 13 is a flowchart illustrating the coding/decoding method according to the fourth embodiment of the present invention.

Meanwhile, it is hereinafter described in order of operation of the imaging device 3B and operation (decoding method) of the decoding device 4B for the purpose of description.

The operation of the imaging device 3B according to the fourth embodiment is different from the operation of the imaging device 3 described in the above-described first embodiment only in that step S21 is added and steps S2B and S3B are added in place of steps S2 and S3, respectively.

Therefore, only steps S21, S2B, and S3B are hereinafter described.

Step S21 is performed after step S1.

At step S21, the sorting unit 315B sorts the moving image frame sequence captured by the imaging element 312 to be Gray coded through a signal processing unit 313 and a Gray coding unit 314 into the sub frames FR, FB, FGr, and FGb (sorts according to the types of the corresponding pixel groups) for each frame.

After step S21, the sorting unit 315B sorts the sub frames FR, FB, FGr, and FGb sorted at step S21 into the key lines (red pixel key line, blue pixel key line, first green pixel key line, and second green pixel key line) and the non-key lines (red pixel non-key line, blue pixel non-key line, first green pixel non-key line, and second green pixel non-key line), outputs the key lines to the transmitting unit 33, and outputs the non-key lines to the coding units 321R, 321B, 321Gr, and 321Gb (step S2B: sorting step).

After step S2B, the coding units 321R, 321B, 321Gr, and 321Gb to which the non-key lines sorted at step S2B are input perform the coding processes to the non-key lines in parallel (step S3B: coding step).

The decoding method according to the fourth embodiment is different from the decoding method described in the above-described first embodiment only in that steps S6B and S7B are added in place of steps S6 and S7.

Therefore, only steps S6B and S7B are hereinafter described.

Step S6B is performed after step S5.

At step S6B, the decoding units 431R, 431B, 431Gr, and 431Gb perform the decoding processes to the non-key lines after the coding process at step S3B in parallel. Meanwhile, contents of each decoding process are similar to those of the decoding process (step S6) described in the above-described first embodiment except that the information used for calculating the first and second log-likelihood ratios t_{j,0} and q_{i,0} serving as the initial values is different as described above.

After step S6B, the error detecting unit 432B performs the parity check on each non-key line (target line) estimated by each decoding process by each decoding unit 431 (step S7B) to detect whether there is an error (step S8).

The fourth embodiment of the present invention described above has the following effect in addition to the effect similar to that of the above-described first embodiment.

In the fourth embodiment, the imaging system 1B sorts the image data according to the types of the corresponding pixel groups and performs the coding process and the decoding process for each type of the corresponding pixel group. Therefore, it is possible to use the different low-density parity check matrix used for the coding process for each type of the corresponding pixel group, thereby improving a degree of freedom of the decoding process. Correlation between the corresponding pixel groups of the same type is higher than that of the different types in one frame, so that it is possible to estimate the non-key line before the coding process with a significantly high degree of accuracy by performing the coding process and the decoding process for each type of the corresponding pixel group.

Meanwhile, although the configuration in which the configuration to perform the coding process and the decoding process for each type of the corresponding pixel group is applied to the above-described first embodiment is described in the above-described fourth embodiment, there is no limitation, and this may also be applied to the above-described second and third embodiments.

### Fifth Embodiment

A fifth embodiment of the present invention is next described.

In the following description, the same reference sign is assigned to a configuration and a step similar to those of the above-described first embodiment and detailed description thereof is omitted or simplified.

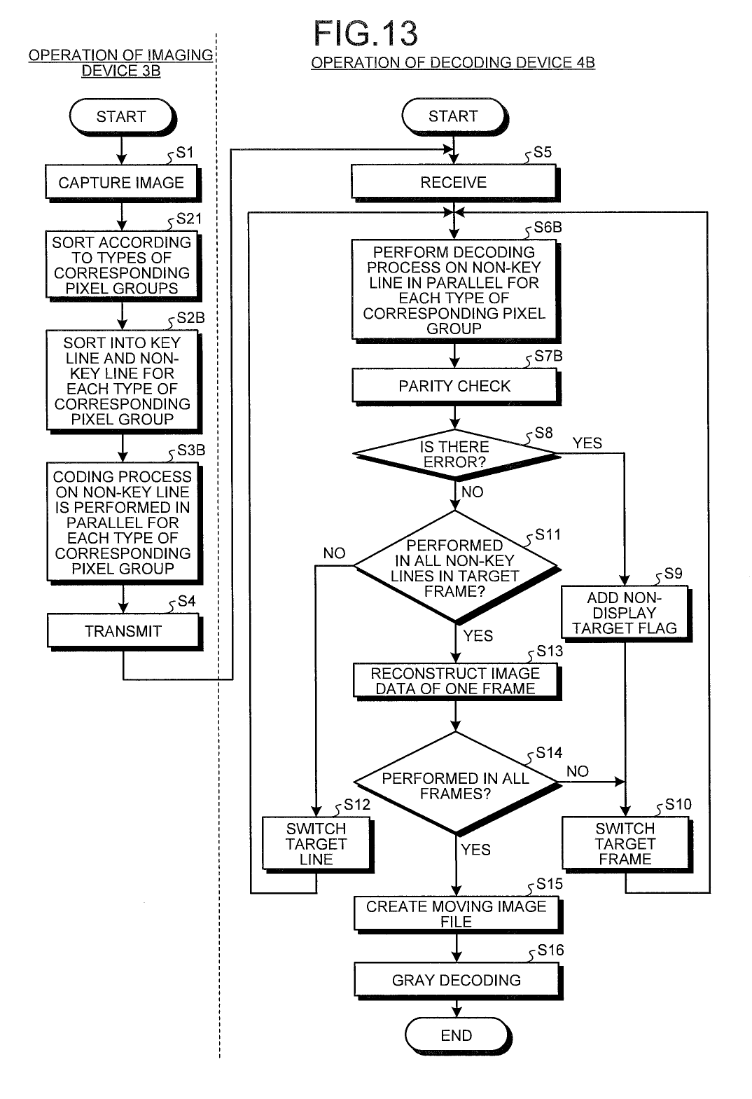

FIG. 14 is a schematic diagram illustrating a capsule endoscope system 10 according to the fifth embodiment of the present invention.

In the fifth embodiment, an imaging system 1 described in the above-described first embodiment is applied to the capsule endoscope system 1C.

The capsule endoscope system 10 is a system which obtains an in-vivo image in a subject 100 by using a swallow type capsule endoscope 3C. The capsule endoscope system 10 is provided with a receiving device 5, a decoding device 4C, a movable recording medium 6 and the like in addition to the capsule endoscope 3C as illustrated in FIG. 14.

The recording medium 6 is a movable recording medium for transferring data between the receiving device 5 and the decoding device 4C formed so as to be attachable/detachable to/from the receiving device 5 and the decoding device 4C.

The capsule endoscope 3C is a capsule type endoscopic device formed to have a size insertable into an organ of the subject 100 and has a function and a configuration similar to those of an imaging device 3 described in the above-described first embodiment (imaging unit 31, control unit 32, and transmitting unit 33).

Specifically, the capsule endoscope 3C is inserted into the organ of the subject 100 through oral intake and the like and sequentially captures in-vivo images (at a frame rate of 30 images per second, for example) while moving in the organ by peristalsis and the like.

The capsule endoscope 3C sorts image data generated by capturing into a key line and a non-key line for each frame as in the imaging device 3 described in the above-described first embodiment. The capsule endoscope 3C does not code the key line but performs a coding process on the non-key line and makes the key line and the non-key line a data stream to transmit.

The receiving device 5 provided with a plurality of receiving antennas 5a to 5h receives moving image data (data stream) from the capsule endoscope 3C in the subject 100 through at least one of a plurality of receiving antennas 5a to 5h. The receiving device 5 stores the received moving image data in the recording medium 6 inserted into the receiving device 5.

Meanwhile, the receiving antennas 5a to 5h may be arranged on a body surface of the subject 100 as illustrated in FIG. 14 or may be arranged on a jacket which the subject 100 wears. The number of receiving antennas provided on the receiving device 5 may be one or more and is not especially limited to eight.

FIG. 15 is a block diagram illustrating the decoding device 4C according to the fifth embodiment of the present invention.

The decoding device 4C formed as a work station which obtains the moving image data in the subject 100 and decodes the obtained moving image data has a function and a configuration (memory unit 42 and control unit 43) substantially similar to those of a decoding device 4 described in the above-described first embodiment as illustrated in FIG. 15. The decoding device 4C is provided with a reader/writer 44, an input unit 45 such as a keyboard and a mouse, a display unit 46 such as a liquid crystal display and the like in addition to the memory unit 42 and the control unit 43.

The reader/writer 44 retrieves the moving image data stored in the recording medium 6 under control of the control unit 43 when the recording medium 6 is inserted into the reader/writer 44. That is to say, the reader/writer 44 serves as a data obtaining unit according to the present invention.

The reader/writer 44 transfers the retrieved moving image data to the control unit 43. The moving image data transferred to the control unit 43 is stored in the memory unit 42.

The control unit 43 performs a decoding process and the like to create the moving image file as in the decoding device 4 described in the above-described first embodiment. The control unit 43 displays a moving image based on the moving image file (in-vivo image of the subject 100) on the display unit 46 according to input operation on the input unit 45 by a user.

Meanwhile, in the above-described first embodiment, a decoding unit 431 calculates a second log-likelihood ratio q_{i,0} serving as an initial value by using the key line “which comes immediately before” a target line (non-key line) in chronological order in a target frame and performs repeated decoding (hereinafter referred to as repeated decoding in a forward direction) by using the second log-likelihood ratio q_{i,0}.

In contrast, in the fifth embodiment, the decoding unit 431 calculates the second log-likelihood ratio q_{i,0} serving as the initial value by using the key line which comes immediately after the target line (non-key line) in chronological order in the target frame (with a larger row number adjacently located in the target frame) and also performs the repeated decoding (hereinafter referred to as repeated decoding in a trace back direction) by using the second log-likelihood ratio q_{i,0} in addition to the repeated decoding in the forward direction.

Then, the control unit 43 creates the moving image file by using the non-key line determined to have no error by parity check out of the non key-lines estimated after the repeated decoding in the forward direction and in the trace back direction. Herein, when it is determined that both the non-key lines estimated after the repeated decoding in the forward direction and in the trace back direction have no error or have an error by the parity check, any non-key line may be adopted.

Meanwhile, it is also possible to create the moving image file by using the non-key line satisfying a condition described in the above-described third embodiment (the number of bits with which an absolute value of a posteriori log-likelihood ratio is smaller than a first threshold is larger than a second threshold) out of the non-key lines estimated after the repeated decoding in the forward direction and in the trace back direction. Herein, when both the non-key lines estimated after the repeated decoding in the forward direction and in the trace back direction satisfy the above-described condition or do not satisfy the above-described condition, any non-key line may be adopted.

The fifth embodiment of the present invention described above has the following effect in addition to the effect similar to that of the above-described first embodiment.

In the fifth embodiment, the decoding unit 431 performs the repeated decoding in both the forward direction and the trace back direction.

For example, suppose a case in which a ratio between the key lines and the non-key lines is 1:3 in one frame, that is to say, there is one key line in each four blocks (for example, a case illustrated in FIG. 2).

In this case, the non-key lines in the third and fourth rows are located closer to the key lines in the first and second rows (key lines “which come immediately before” in chronological order) than the key lines in ninth and tenth rows (key lines “which come immediately after” in chronological order) as illustrated in FIG. 2, so that they have higher correlation with the former. Similarly, the non-key lines in seventh and eighth rows are located closer to the key lines in the ninth and tenth rows (key lines “which come immediately after” in chronological order) than the key lines in the first and second key lines (key lines “which come immediately before” in chronological order), so that they have higher correlation with the former.

Therefore, it is possible to estimate the non-key lines in the third and fourth rows with a high degree of accuracy by the repeated decoding in the forward direction using the key lines “which come immediately before” in chronological order having high correlation. It is possible to estimate the non-key lines in the seventh and eighth rows with a high degree of accuracy by the repeated decoding in the trace back direction using the key lines “which come immediately after” in chronological order having high correlation.

Therefore, it is possible to estimate all the non-key lines in one frame with a high degree of accuracy and create the moving image file such that deterioration in image quality thereof from the moving image data generated by the capsule endoscope 3C is inhibited.

Meanwhile, although the imaging system 1 described in the above-described first embodiment is applied to the capsule endoscope system 10 in the above-described fifth embodiment, it is also possible to apply imaging systems 1, 1A, and 1B described in the above-described second to fourth embodiments to the capsule endoscope system. The imaging system according to the present invention may also be applied to another system. For example, the imaging system according to the present invention may also be applied to a monitoring camera system provided with a monitoring camera serving as the imaging device according to the present invention and the decoding device according to the present invention.

A configuration in which the receiving device 5 has the function and configuration of the decoding device 4 (memory unit 42 and control unit 43) described in the above-described first embodiment is also possible in the above-described fifth embodiment.

Furthermore, although the decoding device 4C serving as the work station has the function as the decoding device according to the present invention in the above-described embodiment, there is no limitation. For example, an external cloud computer is allowed to have the function as the decoding device according to the present invention, the moving image data from the capsule endoscope 3C received by the receiving device 5 is transmitted to the cloud computer, and the moving image data is decoded by the cloud computer. The cloud computer codes the decoded moving image data to JPEG/MPEG coded data easily decoded by user equipment and distributes the same to the user.

### Another Embodiment

Meanwhile, although each of imaging devices 3 and 3B (capsule endoscope 3C) performs a coding process on all bit sequences of Gray codes in all pixel positions included in a non-key line in the above-described first to fifth embodiment, there is no limitation. For example, it is also possible to configure such that low-order bits of all the bit sequences of the Gray codes in all the pixel positions included in the non-key line are thinned out and the coding process is performed on the non-key line after the thinning out (coding process is performed on a part of the non-key line).

When the imaging devices 3 and 3B (capsule endoscope 3C) are configured in the above-described manner, a configuration to interpolate the thinned bits on a side of decoding devices 4, 4A to 4C may be added.

Although a function to perform the coding process and a function to perform a decoding process are formed by software in the above-described first to fifth embodiments, the present invention is not limited to this, and the functions may be formed by hardware.

Although the coding process is not performed on the key-line in the above-described first to fifth embodiments, there is no limitation, and an error-correcting code may be put into the key line.

Although a key block and a non-key block according to the present invention are a plurality of pixels arranged in a row direction in the above-described first to fifth embodiments, there is no limitation, and they may be a plurality of pixels arranged in a column direction or a plurality of pixels arranged in positions separated from one another.

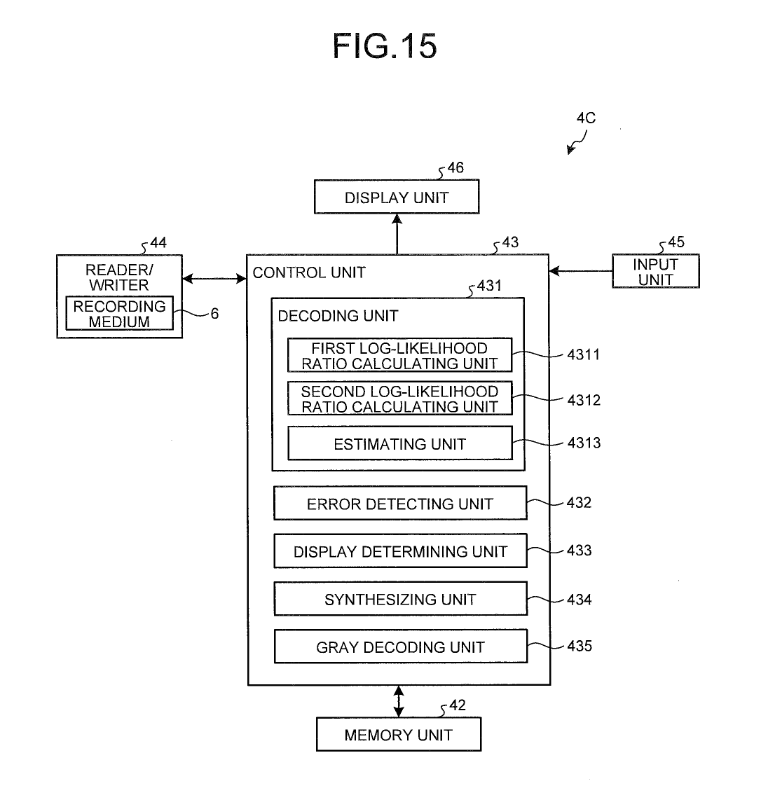

FIG. 16 is a view illustrating a variation of the first to fifth embodiments of the present invention.

Although each of the imaging devices 3 and 3B (capsule endoscope 3C) sequentially generates image data by capturing and performs the coding process on all frames (non-key lines) in the above-described first to fifth embodiments, there is no limitation. For example, it is also possible to configure such that the coding process is performed only on a part of frames of a plurality of generated image data as illustrated in FIG. 16.

Hereinafter, the frame on which the coding process is not performed is referred to as a key frame F^{K} (FIG. 16) and the frame on which the coding process is performed is referred to as a non-key frame F^{S} (FIG. 16).

In the example in FIG. 16, one out of three frames is made the key frame F^{K} and the others are made the non-key frames F^{S}.

When it is configured in such a manner, even when an error is detected by parity check on the non-key line (steps S7 and S7B) and when the non-key line is determined to be no good in a determining process based on a posteriori log-likelihood ratio of the non-key line restored by repeated decoding (steps S19 and S20), it becomes possible to predict the non-key line by using the key frame F^{K} which comes immediately before or after the non-key frame F^{S} including the non-key line in chronological order.

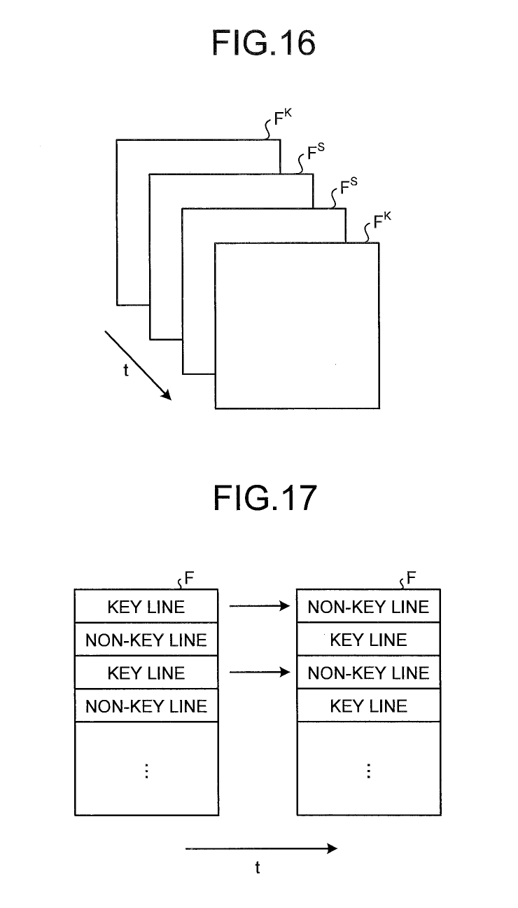

FIG. 17 is a view illustrating a variation of the first to fifth embodiments of the present invention.

In the above-described first to fifth embodiments, it is also possible to set such that the key lines and the non-key lines in an image F of one frame are alternately arranged in a vertical direction as illustrated in FIG. 17. At that time, when it is set such that positions of the key lines and positions of the non-key lines are alternately arranged between the frames adjacent to each other in chronological order as illustrated in FIG. 17, the following effect is obtained.

That is to say, even when an error is detected by the parity check on the non-key line (steps S7 and S7B) or when the non-key line is determined to be no good in the determining process based on the posteriori log-likelihood ratio of the non-key line restored by the repeated decoding (steps S19 and S20), it is possible to predict the non-key line by using the key line in the same position as the non-key line in the frame which comes immediately before or after the frame including the non-key line in chronological order.

According to some embodiments, the following configuration may be adopted as an imaging device used in combination with the decoding device.

That is to say, the imaging device does not code a key block but performs a coding process on at least a part of a non-key block out of image data generated by capturing. Then, the imaging device transmits the key block and non-key block. Therefore, it is possible to decrease an information amount of the image data to be transmitted.

The decoding device according to some embodiments performs repeated decoding by a belief-propagation method based on a first log-likelihood ratio obtained from the non-key block on at least a part of which the coding process is performed and a second log-likelihood ratio obtained from the key block which is not coded and characteristic information. Therefore, it is possible to adopt a simple coding system as the coding system performed by the imaging device.

From above, there is an effect that the decoding device capable of inhibiting a load and consumed power of the imaging device even when a frame rate at the time of imaging is made larger may be realized.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A decoding device configured to decode image data coded by an imaging device, the decoding device comprising:
a data obtaining unit configured to obtain a key block and a non-key brock, the key block forming a part of the image data of one frame generated by the imaging device, and the non-key block forming a part of the image data of one frame generated by the imaging device on at least a part of which a coding process is performed;
a characteristic information storage unit configured to store characteristic information regarding a pixel value correlation characteristic in a frame; and
a decoding unit configured to perform repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and the characteristic information stored in the characteristic information storage unit.

2. The decoding device according to claim 1, wherein
the characteristic information storage unit is configured to store a plurality of pieces of different characteristic information and
the decoding device is configured to change the second log-likelihood ratio to a second log-likelihood ratio obtained from the key block and the characteristic information different from the previously used characteristic information out of the plurality of pieces of characteristic information and re-perform the repeated decoding by using the changed second log-likelihood ratio.

3. The decoding device according to claim 1, wherein the decoding unit is configured to perform the repeated decoding in a forward direction and the repeated decoding in a trace back direction, the repeated decoding in the forward direction being the repeated decoding performed based on the first log-likelihood ratio and the second log-likelihood ratio obtained from the key block obtained immediately before the non-key block in chronological order by the data obtaining unit and the characteristic information, and the repeated decoding in the trace back direction being the repeated decoding performed based on the first log-likelihood ratio and the second log-likelihood ratio obtained from the key block obtained immediately after the non-key block in chronological order by the data obtaining unit and the characteristic information.

4. The decoding device according to claim 3, further comprising:
an error detecting unit configured to detect whether there is an error by performing parity check on the non-key block estimated after the repeated decoding by the decoding unit, wherein
the decoding unit is configured to output the non-key block estimated after the repeated decoding in the forward direction or the non-key block estimated after the repeated decoding in the trace back direction as a decoded result based on a detection result by the error detecting unit.

5. The decoding device according to claim 3, wherein the decoding unit is configured to output the non-key block estimated after the repeated decoding in the forward direction or the non-key block estimated after the repeated decoding in the trace back direction as a decoded result based on a posteriori log-likelihood ratio of the non-key block restored by the repeated decoding in the forward direction and a posteriori log-likelihood ratio of the non-key block restored by the repeated decoding in the trace back direction.

6. The decoding device according to claim 1, further comprising: a display determining unit configured to perform a process of determining whether the non-key block estimated after the repeated decoding by the decoding unit is made a display target.

7. The decoding device according to claim 6, further comprising:
the error detecting unit configured to detect whether there is an error by performing the parity check on the non-key block estimated after the repeated decoding by the decoding unit, wherein
the display determining unit is configured to perform the determining process based on a detection result by the error detecting unit.

8. The decoding device according to claim 6, wherein the display determining unit is configured to perform the determining process based on a posteriori log-likelihood ratio of the non-key block restored by the repeated decoding by the decoding unit.

9. The decoding device according to claim 1, wherein each of the key block and the non-key block is a pixel group on at least one line arranged in a row direction or a column direction in the frame.

10. The decoding device according to claim 1, wherein
the imaging device includes an imaging unit including an imaging element and a color filter on which a plurality of filter groups grouped according to wavelength bands of transmitted light is arranged in a specified format on a light receiving surface of the imaging element,
the imaging unit is configured to generate image data corresponding to incident light through the color filter and sort the image data into the key block and the non-key block for each frame, and
the decoding unit is configured to group pixels included in the non-key block according to groups of the plurality of filter groups and perform the repeated decoding for each group.

11. The decoding device according to claim 1, wherein
the imaging device includes an imaging unit including an imaging element and a color filter on which a plurality of filter groups grouped according to wavelength bands of transmitted light is arranged in a specified format on a light receiving surface of the imaging element,
the imaging unit is configured to generate image data corresponding to incident light through the color filter and sort the image data into the key block and the non-key block for each frame,
the characteristic information storage unit is configured to store a plurality of pieces of characteristic information for each group of the plurality of filter groups, and
the decoding unit is configured to group the pixels included in the non-key block according to groups of the plurality of filter groups and perform the repeated decoding for each group by using the characteristic information corresponding to a group of the pixels on which the repeated decoding is performed out of the plurality of pieces of characteristic information stored in the characteristic information storage unit.

12. An imaging system comprising:
an imaging device configured to code image data generated by capturing an image of a subject to transmit, and
a decoding device configured to receive the coded image data to decode, wherein
the imaging device includes:
an imaging unit configured to generate image data corresponding to incident light and sort the image data into a key block and a non-key block for each frame;
a coding unit configured to perform a coding process on at least a part of the non-key block; and
a transmitting unit configured to transmit the key block and the non-key block on at least a part of which the coding process is performed, and
the decoding device includes:
a receiving unit configured to receive the key block and the non-key block on at least a part of which the coding process is performed;
a characteristic information storage unit configured to store characteristic information regarding a pixel value correlation characteristic for each color in a frame; and
a decoding unit configured to perform repeated decoding by a belief-propagation method based on a first log-likelihood ratio and a second log-likelihood ratio to estimate the non-key block before coding process, the first log-likelihood being obtained from the non-key block on at least a part of which the coding process is performed, and the second log-likelihood ratio being obtained from the key block and the characteristic information stored in the characteristic information storage unit.

13. The imaging system according to claim 12, wherein the coding process is syndrome coding using a parity check matrix.

14. The imaging system according to claim 12, wherein
the imaging unit includes:
an imaging element; and
a color filter in which a plurality of filter groups grouped according to wavelength bands of transmitted light is arranged in a specified format on a light receiving surface of the imaging element, and
the coding unit is configured to group pixels included in the non-key block according to groups of the plurality of filter groups and perform the coding process by using a coding arithmetic matrix on at least a part of a group for each group, the coding arithmetic matrix used for at least one group out of the grouped plurality of groups being different from the coding arithmetic matrix used for other groups.

15. The imaging system according to claim 12, wherein the imaging device is a capsule endoscope insertable into a subject.

16. A decoding method executed by a decoding device configured to decode image data coded by an imaging device, the decoding method comprising:
obtaining a key block and a non-key block, the key block forming a part of the image data of one frame generated by the imaging device, and the non-key block forming a part of the image data of one frame generated by the imaging device on at least a part of which a coding process is performed; and
performing repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic in a frame.

17. A coding and decoding method executed by an imaging system including an imaging device configured to code image data generated by capturing an image of a subject to transmit and a decoding device configured to receive the coded image data to decode, the coding and decoding method comprising:
generating, by the imaging device, image data corresponding to incident light;
sorting, by the imaging device, the image data into a key block and a non-key block for each frame;
performing, by the imaging device, a coding process on at least a part of the key block;
transmitting, by the imaging device, the key block and the non-key block on at least a part of which the coding process is performed;
receiving, by the decoding device, the key block and the non-key block on at least a part of which the coding process is performed; and
performing, by the decoding device, repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic for each color in a frame.

18. A non-transitory computer-readable recording medium having an executable program recorded thereon, the program instructing a processor, which is included in a decoding device configured to decode image data coded by an imaging device, to execute:
obtaining a key block and a non-key brock, the key block forming a part of the image data of one frame generated by the imaging device, and the non-key block forming a part of the image data of one frame generated by the imaging device on at least a part of which a coding process is performed; and
performing repeated decoding by a belief-propagation method based on a first log-likelihood ratio and a second log-likelihood ratio to estimate the non-key block before the coding process, the first log-likelihood being obtained from the non-key block on at least a part of which the coding process is performed, and the second log-likelihood ratio being obtained from the key block and characteristic information regarding a pixel value correlation characteristic in a frame.
